SEQUENCE LISTING (13 sequences)

SEQ ID NO: 1 - protein, 211 aa, Artificial Sequence
  Description of Artificial Sequence Synthetic Polypeptide
AYSNTYQEFTNIDQAKAWGNAQYKKYGLSKSEKEAIVSYTKSASEINGKLRQNKGVINGFPSNLIKQVELLDKSFNKMKTPENIMLFRGDDPAYLGTEFQNTLLNSNGTINKTAFEKAKAKFLNKDRLEYGYISTSLMNVSQFAGRPIITKFKVAKGSKAGYIDPISAFAGQLQMLLPRHSTYHIDDMRLSSDGKQIIITATMMGTAINPK

SEQ ID NO: 2 - protein, 211 aa, Artificial Sequence
  Description of Artificial Sequence Synthetic Polypeptide
AYSNTYQEFTNIDQAKAWGNAQYKKYGLSKSEKEAIVSYTKSASEINGKLRQNKGVINGFPSNLIKQVELLDKSFNKMKTPENIMLFRGDDPAYLGTEFQNTLLNSNGTINKTAFEKAKAKFLNKDRLEYGYISTSLMNVSQFAGRPIITKFKVAKGSKAGYIDPISAFAGQLAMLLPRHSTYHIDDMRLSSDGKQIIITATMMGTAINPK

SEQ ID NO: 3 - protein, 98 aa, Artificial Sequence
  Description of Artificial Sequence Synthetic Polypeptide
AYSNTYQEFTNIDQAKAWGNAQYKKYGLSKSEKEAIVSYTKSASEINGKLRQNKGVINGFPSNLIKQVELLDKSFNKMKTPENIMLFRGDDPAYLGTE

SEQ ID NO: 4 - protein, 115 aa, Artificial Sequence
  Description of Artificial Sequence Synthetic Polypeptide
TEFQNTLLNSNGTINKTAFEKAKAKFLNKDRLEYGYISTSLMNVSQFAGRPIITKFKVAKGSKAGYIDPISAFAGQLEMLLPRHSTYHIDDMRLSSDGKQIIITATMMGTAINPK

SEQ ID NO: 5 - protein, 63 aa, Artificial Sequence
  Description of Artificial Sequence Synthetic Polypeptide
SNLIKQVELLDKSFNKMKTPENIMLFRGDDPAYLGTEFQNTLLNSNGTINKTAFEKAKAKFLN

SEQ ID NO: 6 - protein, 52 aa, Artificial Sequence
  Description of Artificial Sequence Synthetic Polypeptide
AYSNTYQEFTNIDQAKAWGNAQYKKYGLSKSEKEAIVSYTKSASEINGKLRQ

SEQ ID NO: 7 - protein, 48 aa, Artificial Sequence
  Description of Artificial Sequence Synthetic Polypeptide
RQNKGVINGFPSNLIKQVELLDKSFNKMKTPENIMLFRGDDPAYLGTE

SEQ ID NO: 8 - protein, 50 aa, Artificial Sequence
  Description of Artificial Sequence Synthetic Polypeptide
SKSEKEAIVSYTKSASEINGKLRQNKGVINGFPSNLIKQVELLDKSFNKM

SEQ ID NO: 9 - protein, 62 aa, Artificial Sequence
  Description of Artificial Sequence Synthetic Polypeptide
EFQNTLLNSNGTINKTAFEKAKAKFLNKDRLEYGYISTSLMNVSQFAGRPIITKFKVAKGSK

SEQ ID NO: 10 - protein, 53 aa, Artificial Sequence
  Description of Artificial Sequence Synthetic Polypeptide
KAGYIDPISAFAGQLEMLLPRHSTYHIDDMRLSSDGKQIIITATMMGTAINPK

SEQ ID NO: 11 - protein, 45 aa, Artificial Sequence
  Description of Artificial Sequence Synthetic Polypeptide
MNVSQFAGRPIITKFKVAKGSKAGYIDPISAFAGQLEMLLPRHST

SEQ ID NO: 12 - protein, 17 aa, Artificial Sequence
  Description of Artificial Sequence Synthetic Polypeptide
HSTYHIDDMRLSSDGKQ

SEQ ID NO: 13 - protein, 14 aa, Artificial Sequence
  Description of Artificial Sequence Synthetic Polypeptide
AYSNTYQEFTNIDQ

(19) United States
(12) Patent Application Publication
(10) Pub. No.: US 2005/0255543 A1
(43) Pub. Date: Nov. 17, 2005

(54) Enzyme-deficient c3 botulinum protein species and their use to promote neuronal growth and neuronal regeneration

(76) Inventors: Ingo Just, Hannover (DE); Fred Hofmann, Hannover (DE); Gudrun Ahnert-Hilger, Berlin (DE); Gisela Grosse, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/493,864

(22) PCT Filed: Oct. 28, 2002
(86) PCT No.: PCT/EP02/12039

§ 371 Date: Dec. 15, 2004

(30) Foreign Application Priority Data
Oct. 29, 2001 (DE) 101 54 685.8

(51) Int. Cl. C07H 21/04; C12N 1/21; C12P 21/06; C12N 15/74; C07K 14/33

(52) U.S. Cl. 435/69.1; 435/252.3; 435/471; 530/350; 536/23.7

(57) ABSTRACT

The present invention is directed to nucleotide sequences and their encoded enzyme-deficient proteins and peptides which regulate neurite especially axonal growth, recognitions agents thereto, and the therapeutic and diagnostic uses of such proteins and peptides. The invention relates also to the use of enzyme-deficient or enzymatic inactive C3 proteins/peptide-derivatives derived from Clostridium botulinum for regulation of the growth, expansion and/or differentiation of neurons and neuronal stem cells as well as the regeneration of neurons. In a specific embodiment of the invention, proteins and peptides may be used to promote the regeneration of neuronal axons over long distances following spinal cord damage. The proteins and peptides allow neurite especially axonal outgrowth—without actions on glial cells—in nervous system tissue in vivo and in vitro. They may have important uses in the treatment of central nervous system damage and degenerative nerve diseases.

The present invention is directed to nucleotide sequences and their encoded enzyme-deficient proteins and peptides which regulate neurite especially axonal growth, recognitions agents thereto, and the therapeutic and diagnostic uses of such proteins and peptides. The invention relates also to the use of enzyme-deficient or enzymatic inactive C3 proteins/peptide-derivatives derived from Clostridium botulinum for regulation of the growth, expansion and/or differentiation of neurons and neuronal stem cells as well as the regeneration of neurons. In a specific embodiment of the invention, proteins and peptides may be used to promote the regeneration of neuronal axons over long distances following spinal cord damage. The proteins and peptides allow neurite especially axonal outgrowth—without actions on glial cells—in nervous system tissue in vivo and in vitro. They may have important uses in the treatment of central nervous system damage and degenerative nerve diseases.

Neuronal proliferation, growth, and path-finding of axons as well as the majority of synaptogenesis is mostly completed during the pre- and postnatal development. Besides neurons glial cells are the second cell group in the nervous system. Both neurons and the variety of glial cells derive from a common embryonic origin the neuroectoderm. Whereas neurons build up the communicating system, glial cells have a variety of supportive effects. Development, regeneration and function of the nervous system rely on the controlled interaction of glial cells and neurons. Mature neurons are thought to have no ability to proliferate. Developmental processes and survival of neurons depend on a great variety of molecules which perform their action by the interference with specific neurotrophic factor-receptors and other surface molecules of the neuronal membrane. In adult brain, changes within neuronal networks are more subtle and mostly rely on existing paths and connections which are modulated according to the various external requirements. In this respect, damage of neurons by mechanical injury like the disruption of spinal cord motor neurons or the damage of central neurons following pathological processes, such as Morbus Parkinson, Alzheimer or stroke events, dramatically impairs neuronal function at the level of the individual neuron as well as the neuronal network or the dependent system. Physical damage or pathophysiological processes also impair the balance between neurons and glial cells. Especially during regeneration proliferation of glial cells may hinder the growing axon to find its target. This process is also well known as astrogliosis.

The neurotrophic factors or neurotrophins so far described promote or sustain neuronal development and survival. They include brain-derived neurotrophic factor (BDNF), nerve growth factor (NGF), and the neurotrophins 3 (NT3) and 4/5 (NT4/5). Neurotrophins are probably synthesized as precursors which are cleaved by neuron-specific proteases thereby allowing an individual regulation.

Neurotrophins act via two types of cell surface molecules: the receptor tyrosine kinases trkA, trkB and trkC and the p75 neurotrophin receptor. Whereas binding of neurotrophin to tyrosine kinase receptors promotes neurite outgrowth, activation of p75 appears to be also involved in apoptotic processes of neurons (for review see Lee et al., 2001). It has been reported that p75 acts as a constitutive activator of the small GTPase Rho and that binding of neurotrophins, BDNF, NGF or NT3 or 4 inhibits activation of Rho. Besides the neurotrophic receptors there are other surface molecules like neuropilin 1 and 2 which cause repulsion of neurites via their ligands, the semaphorins and the ephrin receptors which appear to be responsible for the specific guidance of axons by binding to ephrins resulting in the development of neuronal topographic maps. Finally, lysophosphatidic acid (LPA) promotes neurite retraction through a G12/13-initiated activation of Rho. LPA has been shown to signal a broad spectrum of vital cellular events amongst them mitogenesis, platelet aggregation, escape from apoptosis and also as mentioned above neurite retraction. The effects of LPA are mediated by G-protein coupled receptors termed Edg (Endothelial differentiation gene), some of them are also expressed in brain. LPA may be secreted by a variety of cells including post-mitotic neurons as an autocrine or paracrine mediator. It may be also released during mechanical injury, stroke or other pathological processes in brain. Recently, it has been shown that LPA can be dephosphorylated by integral plasma membrane proteins acting as ecto-phosphorylase enzymes. These lipid phosphate phosphohydrolyses (LPPs) comprise a growing number of proteins in various tissue which may also antagonize LPA-effects and sustain axonal growth by dephosphorylating LPA. In a concerted action all these effectors and lysophospholipids, through their specific receptors, or by LPPs add to the development, sustenance and regeneration but also to pathalogical processes in nervous tissue.

Proteins derived from bacteria are highly specific for interacting with defined processes as mentioned above. Amongst these clostridial and other C3-like ADP-ribosyltransferases have been shown to exert their effects by ADP-ribosylation and thus inactivation of the Rho proteins. C3 toxins are considered as exoenzymes their cellular uptake occuring unspecifically through pinocytosis. Alternatively mechanical procedures like the trituration of neurons have to be applied to allow the toxin to interact with its intracellular target the Rho proteins.

Clostridium botulinum C3 is the prototype of the family of C3-like ADP-ribosyltransferases which modify GTPases of the Rho subfamily. C3 cleaves the ubiquitous co-substrate NAD+ and transfers the ADP-ribose moiety to asparagine-41 (Asn-11) of Rho; the ribose is N-glycosidically bound. Asn-41 is located in the so-called effector region of Rho, whose function is coupling with signal transduction proteins to amplify and transmit the signal downstream. In addition to various isoforms of C3 produced by Clostridium botulinum strains, C3-related enzymes are also produced by Clostridium limosum, Bacillus cereus, and Staphylococcus aureus. The C3-like exoenzymes are single-chained peptides with a molecular mass of about 24 kDa and an isoelectric point of 9-10. They are released by the bacteria. In contrast to other toxins, especially the clostridial neurotoxin C1 and the C2 toxin, C3 is devoid of a cell entry machinery. Cell entry into cultured cells is only observed in the presence of high extracellular concentrations of C3 (μM range) and is thought to take place by pinocytosis. Due to lack of extracellular protein substrates and co-substrates, C3 is thought to only reveal intracellular activity. The C3 transferases share their catalytic amino acid glutamate (postion 174 in C3-bot) with all the other ADP-ribosyltransferases; exchange of this glutamate results in enzymatic-inactive C3 proteins.

C3 produced by clostridia and bacilli selectively ADP-ribosylates the Rho isoforms A, B and C but not other members of the Rho or Ras superfamily. Only under special conditions, such as the presence of low concentrations of sodium dodecyl sulphate, the Rac protein is modified to a minor extent. A C3 isoform produced by pathogenic Staphylococcus aureus, designated C3stau, ADP-ribosylates in addition to RhoA, B and C the Rnd or RhoE protein. The RhoE protein also belongs to the Rho subfamily, but it is devoid of a GTP-hydrolysing activity; it seems to be a functional antagonist of Rho, leading to depolymerisation of actin filaments. The major toxic effect of all C3 toxins is the disaggregation of the actin cytoskeleton.

The depolymerisation of stress fibres results in changes in cell shape and cell motility as well as in opening of tight junctions. Cultured cells round up and show a neuron-like neurite-like morphology due to formation of neurite-like retraction fibres. The effect on the tight junctions results in a loss of the barrier function of epithelial and endothelial cell layers. On cultured cell lines these effects can easily be detected and have led to the designation of C3 toxins as cytotoxins.

The cellular effects of C3 can only be interpreted as a result of inactivation of cellular Rho by ADP-ribosylation. Because the bulky and strongly negatively charged ADP-ribose resides in the effector region, it was thought that ADP-ribosylation blocks effector coupling thereby inhibiting downstream signalling.

Because of its confined substrate protein specificity, C3 is an established tool in cell biological research to study the involvement of Rho in cellular functions. The disadvantage of poor cell entry has been overcome by different approaches: I. Microinjection of C3. II. Electroporation of cells in the presence of C3. III. Permeabilisation by digitonin or streptolysin O. IV. Intracellular expression of C3. V. Using chimeric toxins which recruit the cell entry machinery of other toxins; i.e. C3 is fused to enzyme-deficient diphtheria toxin or Clostridium botulinum C2 toxin. All these approaches result in a sufficient intracellular concentration of C3 to cause toxic effects.

The common features of the Rho low-molecular-mass GTP-binding proteins, also called small GTPases, are their molecular mass (20-24 kDa), their C-terminal polyisoprenylation and their property to bind to and hydrolyse guanine nucleotides. They are molecular relays which transmit signals when bound to GTP and stop doing so when bound to GDP. The Rho subfamily comprises Rho, Rac, Cdc42, RhoD, Rnd/RhoE, TC10; the best characterised are Rho, Rac and Cdc42. Rho/Rac/Cdc42-dependent signal pathways are stimulated by receptor-tyrosine kinases (PDGF for Rac) and by G-protein-coupled receptors (LPA for Rho, bradykinin for Cdc42). Stimulation of the Rho signalling starts with the guanine nucleotide exchange factors (GEF), which catalyse the exchange of nucleotides resulting in binding of GTP to Rho. Binding of GTP induces changes in the conformation of the effector region (covering residues 30-42) which allows interaction of Rho with its so-called effector proteins. Effectors are often serine/threonine kinases which are activated by binding of Rho (e.g. ROK/Rho kinases) to phosphorylate downstream targets. In addition to kinases, Rho effectors comprise also multi-domain proteins without enzymatic activity (rhotekin, rhophilin, WASP) which may serve as nucleus for multi-protein complexes to connect different signalling pathways. Thus, the effector proteins amplify and execute the Rho signals. The downstream signalling is terminated by the GTPase-activating protein (GAP), which strongly enhances the inherent GTP-hydrolysing activity of Rho resulting in inactive GDP-bound Rho, where Rho is kept in the complex with GDI (guanine nucleotide dissociation inhibitor). Rho GTPase are ubiquitous and seems to be essential for everyell type. Because C3 exoenzyme does not discriminate between cell types,intracellular Rho of every cell is target of C3.

The Rho proteins are in general the master regulators of the actin cytoskeleton. The isoforms, however, regulate different aspects: Cdc42 is involved in the formation of filopodia (micro spikes), Rac governs the formation of lamellipodia (ruffes) whereas Rho is reponsible for stress fibres. In addition, they are involved in many actin-dependent processes (such as cell motility, cell adhesion, cell-cell contact), as well as membrane trafficking (endo- and exocytosis, phagocytosis). However, Rho functions are also beyond the regulation of the cytoskeleton: Transcriptional activity is governed via the JNK, p38 and NFkappaB pathway; the cell cycle is regulated (G1-S phase transition) and finally, the formation of reactive oxygen species is stimulated through activation of the NADP oxidase of neutrophils.

Since rho is involved in a great variety of intracellular processes the unselective concentration of C3 toxin in neurons and glial cells to impair rho function may be contradictionary to specific processes required i.e. during axon regeneration. In this respect selective effects restricted to neurons and primarily to specialized neuronal compartments i.e the axon and its growth cone or the somato/dendritic compartment are diserable and will improve therapeutic efforts. As a result, to date, effective treatments for central nervous system (CNS) and peripheral nervous system (PNS) injuries and agents and methods for neuronal growth/regeneration have not been developed. Accordingly, there is a need in the art for compounds and methods that modulate and/or direct neurite and especially axonal outgrowth without effects on glial cells.

Thus, the technical problem underlying the present invention is to provide agents and methods which promote neuronal growth and regeneration.

The present invention solves the problem by providing an isolated nucleotide sequence selected from the group consisting of:
(a) a nucleotide sequence encoding a polypeptide having neurite outgrowth activity comprising a amino acid sequence of SEQ ID NO: 1-13;
(b) nucleotide sequences that are complementary to any of the nucleotide sequences of (a);
(c) a nucleotide sequence differing from the nucleotide sequence as claimed in (a) or (b) in codon sequences due to the degeneracy of the genetic code, whereby said nucleotide sequence encodes a polypeptide having a biological activity indicated in (a);
(d) a nucleotide sequence which specifically hybridize under stringent hybridization conditions to any of the nucleotide sequences of (a), (b) or (c);
(e) a nucleotide sequence of (a), (b), (c), or (d) having a deletion, addition, substitution mutation, whereby said nucleotide sequence encodes a polypeptide having a biological activity indicated in (a).

The nucleotide sequences encoding non-enzymatic C3-related polypeptides/proteins having neurite outgrowth activities. In a preferred embodiment of the invention the isolated nucleotide sequence is a genomic DNA, a cDNA or a RNA. Therefore the invention provides isolated nucleic acids encoding enzyme-deficient—that means without Rho-inactivating effects—C3 botulinum polypeptides/peptides or proteins. The present invention is based on the unique observation that certain enzyme-deficient, not Rho-inactivating, C3 botulinum protein species or neurite outgrowth-promoting polypeptides effectively and efficiently increase differentiation of neuronal cells, including increased neuronal ramification of axons, which has several consequences. First, increasing axonal ramification leads to increased synaptic contacts and neuronal communication, thereby increasing neuronal function and performance. Thus, the present invention is useful for treating diseases or disorders marked by reduction of neuronal ramification and function, including but not limited to Parkinson's disease, amyotrophic lateral sclerosis, Alzheimer's disease, or any other neurodegenerative disease, or physical or toxic damage to brain, spinal or peripheral nerve cells. It is noteworthy that it is the axon that during neuronal development and pathfinding defines the place of contact and induces the assembly of receptors at the postsynaptic site in order to form a functionating synaptic contact. In Hirschsprungs disease as an example the failure of neurons in certain parts of the colon results in severe spasms of the muscle tissue because too many cholinergic receptors are expressed waiting for an axon which has not arrived. Similarly the loss of axonal contact of a motorneuron to the respective muscle causes the cholinergic receptors to spread all over the muscle fiber. Further, the present invention is useful for restoring or optimizing neuronal communication, function or performance. In a embodiment the method for inducing neurite and especially axonal outgrowth in the central nervous system of a patient with damage to the central nervous system comprising administering to the patient the said polypeptides/peptides.

An isolated nucleic acid is present as other than a naturally occurring chromosome or transcript in its natural state and is typically joined in sequence to at least one nucleotide with which it is not normally associated on a natural chromosome. A partially pure nucleotide sequence constitutes at least about 5%, preferably at least about 30%, and more preferably at least about 90% by weight of total nucleic acid present in a given fraction. Unique portions of the disclosed nucleic acids are of length sufficient to distinguish previously known nucleic acids. Thus, a unique portion has a nucleotide sequence at least long enough to define a novel oligonucleotide, usually at least about 5-30 bp in length. The invention also provides for the disclosed nucleic acids modified by transitions, transversions, deletions, insertions, or other modifications such as alternative splicing and also provides for genomic sequences, and gene flanking sequences, including regulatory sequences; included are DNA and RNA sequences, sense and antisense. Preferred DNA sequence portions encode the preferred amino acid sequence portions disclosed above. For antisense applications where the inhibition of expression is indicated, especially useful oligonucleotides are between about 10 and 30 nucleotides in length and include sequences surrounding the disclosed ATG start site, especially the oligonucleotides defined by the disclosed sequence beginning about 5 nucleotides before the start site and ending about 10 nucleotides after the disclosed start site. The polypeptide/peptide encoding nucleic acids can be subject to alternative purification, synthesis, modification, sequencing, expression, transfection, administration or other use by methods disclosed in standard manuals such as Molecular Cloning, A Laboratory Manual (2nd Ed., Sambrook, Fritsch and Maniatis, Cold Spring Harbor), Current Protocols in Molecular Biology (Eds. Aufubel, Brent, Kingston, More, Feidman, Smith and Stuhl, Greene PubI. Assoc., Wiley-lnterscience, New York, N.Y., 1992) or that are otherwise known in the art.

The term polynucleotide is used to mean a polymeric form of nucleotides of any length, which contain deoxyribonucleotides, ribonucleotides, and/or their analogs. The terms polynucleotide and nucleotide as used herein are used interchangeably. Polynucleotides can have any three-dimensional structure, and can perform any function, known or unknown. The term polynucleotide includes double-stranded, single-stranded, and triple-helical molecules. Unless otherwise specified or required, any embodiment of the invention described herein that is a polynucleotide encompasses both the double-stranded form and each of two complementary single-stranded forms known or predicted to make up the double stranded form.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes, and primers. A polynucleotide can be comprised of modified nucleotides, such as methylated nucleotides and nucleotide analogs. Analogs of purines and pyrimidines are known in the art, and include, but are not limited to, aziridinylcytosine, 4-acetylcytosine, 5-fluorouracil, 5-bromouracil, 5-carboxymethylaminomethyl-2-thiouracil, 5-carboxymethyl-aminomethyluracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, pseudouracil, 5-pentynyluracil and 2,6-diaminopurine. The use of uracil as a substitute for thymine in a deoxyribonucleic acid is also considered an analogous form of pyrimidine. If present, modification to the nucleotide structure can be imparted before or after assembly of the polymer. The sequence of nucleotides can be interrupted by non-nucleotide components. A polynucleotide can be further modified after polymerization, such as by conjugation with a labeling component. Other types of modifications included in this definition are, for example, caps, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars can be replaced by phosphonate groups, phosphate groups, protected by standard protecting groups, or activated to prepare additional linkages to additional nucleotides, or can be conjugated to solid supports. The 5′ and 3′ terminal hydroxy groups can be phosphorylated or substituted with amines or organic capping group moieties of from 1 to 20 carbon atoms. Other hydroxyls can also be derivatized to standard protecting groups. Polynucleotides can also contain analogous forms of ribose or deoxyribose sugars that are generally known in the art, including, but not limited to, 2′-O-methyl-, 2′-O-allyl, 2′-fluoro- or 2′-azido-ribose, carbocyclic sugar analogs, .alpha.-anomeric sugars, epimeric sugars such as arabinose, xyloses or lyxoses, pyranose sugars, furanose sugars, sedoheptuloses, acyclic analogs and abasic nucleoside analogs such as methyl riboside. As noted above, one or more phosphodiester linkages can be replaced by alternative linking groups. These alternative linking groups include, but are not limited to, embodiments wherein phosphate is replaced by P(O)S (thioate), P(S)S (dithioate), (O)NR2 (amidate), P(O)R, P(O)OR′, CO or CH.sub.2 (formacetal), in which each R or R′ is independently H or substituted or unsubstituted alkyl (1-20 C) optionally containing and ether (—O—) linkage, aryl, alkenyl, cycloalky, cycloalkenyl or araldyl. Not all linkages in a polynucleotide need be identical. Although conventional sugars and bases will be used in applying the method of the invention, substitution of analogous forms of sugars, purines and pyrimidines can be advantageous in designing a final product, as can alternative backbone structures like a polyamide backbone. An antisense polynucleotide is a sequence complementary to all or part of a functional RNA or DNA. For example, antisense RNA is complementary to sequences of the mRNA copied from the gene. A fragment (also called a region) of a polynucleotide (i.e., a polynucleotide encoding a sarp) is a polynucleotide comprised of at least 9 contiguous nucleotides of the novel genes. Preferred fragments are comprised of a region encoding at least 5 contiguous amino acid residues, more preferably, at least 10 contiguous amino acid residues, and even more preferably at least 15 contiguous amino acid residues. The term recombinant polynucleotide intends a polynucleotide of genomic, cDNA, semisynthetic, or synthetic in origin which, by virtue of its origin or manipulation: is not associated with all or a portion of a polynucleotide with which it is associated in nature; is linked to a polynucleotide other than that to which it is linked in nature; or does not occur in nature.

Stringent conditions for hybridization of both DNA/DNA and DNA/RNA are as described in Sambrook et al. (1989) MOLECULAR CLONING, A LABORATORY MANUAL, 2nd. Ed., Cold Spring Harbor Laboratory Press. Examples of relevant conditions include (in order of increasing stringency): incubation temperatures of 25 degree C., 37 degree C., 50 degree C., and 68 degree C.; buffer concentrations of 10 times SSC, 6 times SSC, 1 times SSC (where SSC is 0.15M NaCl and 15 mM citrate buffer) and their equivalent using other buffer systems; formamide concentrations of 0%, 25%, 50% and 75%; incubation times from 5 minutes to 24 hours; 1, 2, or more washing steps; wash incubation times of 1, 2, or 15 minutes; and wash solutions of 6 times SSC, 1 times SSC, 0.1 times SSC, or deionized water.

The present invention also relates to a vector comprising the isolated nucleotide sequence of the invention; and also relates to a host cell transfected with the vector of the invention. The invention provides a method of producing a polypeptide of the invention comprising the steps of introducing an isolated nucleic acid of the invention or a vector of the invention into a host cell, culturing said host cell under conditions suitable for expression of said polypeptide, and recovering said polypeptide. A large number of vectors, including plasmid and viral vectors, have been described for expression in a variety of eukaryotic and prokaryotic hosts. Advantageously, vectors will often include a promotor operably linked to the polypeptide/peptide-encoding portion, one or more replication systems for cloning or expression, one or more markers for selection in the host, e.g. antibiotic resistance. The inserted coding sequences may be synthesized, isolated from natural sources, prepared as hybrids, etc. Suitable host cells may be transformed/transfected/infected by any suitable method including electroporation, CaCl.sub.2 mediated DNA uptake, viral infection, microinjection, microprojectile, or other methods. Appropriate host cells include bacteria, archebacteria, fungi, especially yeast, and plant and animal cells, especially mammalian cells. Of particular interest are E. coli, B, Subtilis, Sacchammyces cerevisiae, SF9 cells, C129 cells, 293 cells, Neurospora, and CHO, COS, HeLa cells, immortalized mammalian myeloid and lymphoid cell lines, and pluripotent cells, especially mammalian ES cells and zygotes. Preferred expression systems include COS-7, 293, BHK, CHO, CHOp38, BON, PC12, SHSY, C6, F98 TM4, CV1, VERO-76, HELA, MDCK, BRL 3A, W138, Hep G2, MMT 060562, TRI cells, and baculovirus systems. Preferred replication systems include M13, ColE1, SV40, baculovirus, lambda, adenovirus, MV, BPV, etc. A large number of transcription initiation and termination regulatory regions have been isolated and shown to be effective in the transcription and translation of hetemlogous proteins in the various hosts. Examples of these regions, methods of isolation, manner of manipulation, etc. are known in the art. For the production of stably transformed cells and transgenic animals, the subject nucleic acids may be integrated into a host genome by recombination events. For example, such a nucleic acid can be electroporated into a cell, and thereby effect homologous recombination at the site of an endogenous gene, an analog or pseudogene thereof, or a sequence with substantial identity to an peptide/receptor-encoding gene. Other recombination-based methods such as nonhomologous recombinations, deletion of endogenous gene by homologous recombination, especially in pluripotent cells, etc., provide additional applications. Preferred transgenics and stable transformants over-express or under-express (e.g. knock-out cells and animals) a disclosed peptide/receptor gene and find use in drug development and as a disease model. Methods for making transgenic animals, usually rodents, from ES cells or zygotes are known to those skilled in the art.

The present invention also relates to a polypeptide which promotes neurite outgrowth in mammals, selected from the group consisting of:
(a) a polypeptide having an amino acid sequences corresponding to SEQ ID NO: 1-13;
(b) a polypeptide having an amino acid sequence which has at least 40% homology with the amino sequences of SEQ ID NO: 1-13;
(c) a variant of a polypeptide of (a) or (b) containing a plurality of said amino acid sequences;
(d) a conservatively substituted variant of a polypeptide of (a), (b) or (c) comprising a substitution, deletion, and/or insertion of one or more amino acids; or
(e) a functionally equivalent homologue of (a), (b), (c) or (d).

The present invention relates to polypeptides, peptides derived from Clostridium botulinum (C3-bot) and peptidomimetic derivatives that can be used to improve axonal growth (i) without directly interfering with Rho proteins and (ii) without effects on glial cells. C3-bot proteins or polypeptides and peptides can be applied to neurons in nanomolar concentrations avoiding standard procedures such as application of additional proteins for internalisation, adeno-viruses or mechanical treatments to circumvent the plasma membrane barrier. The C3-bot proteins and peptides derived from them which are devoid of enzymatic activity exhibit their neurotrophic effects as extracellular ligands. In addition their effects are restricted to neurons and do not involve actions on glial cells.

The invention arises from the discovery that the C3 proteins or polypeptides/peptides of the invention derived from Clostridium botulinum exert neurotrophic activity in nanomolar concentrations without directly interfering with Rho and without effects on glial cells. In contrast, the well known C3protein of the art from Clostridium botulinum promotes axon growth: (i) when applied at 5-10 times higher concentrations mostly using additional tricks to circumvent the plasma membrane barrier, (ii) with effects on glial cells, and (iii) with a enzymatic/Rho activity.

The terms polypeptide, oligopeptide, peptide and protein are used interchangeably herein to refer to polymers of amino acid residues. The polymer can be linear or branched, it can comprise modified amino acid residues, and it can be interrupted by non-amino acid residues. The terms also encompass an amino acid polymer that has been modified naturally or by intervention; for example, by disulfide bond formation, glycosylation, lipidation, acetylation, phosphorylation, or any other manipulation or modification, such as conjugation with a labeling component. Also included within the definition are, for example, polypeptides containing one or more analogs of an amino acid residue (including, for example, unnatural amino acid residues, etc.), as well as other modifications,—e.g. also peptidomimetic derivates or mimettic peptides—known in the art.

The subject peptides or petidomimetic derivatives may be free or covalently coupled to other atoms or molecules. Frequently the peptides are present as a portion of a larger polypeptide comprising the subject peptide where the remainder of the polypeptide need not be C3 botulinum protein-derived. Alternatively, the subject peptide may be present as a portion of a substantially full-length enzyme-deficient, not Rho-inactivating, C3 botulinum protein which comprises at least about 10, preferably at least about 20, more preferably at least about 40 amino acids of a disclosed protein sequence. The invention provides polypeptides comprising a sequence substantially similar to that of substantially full-length enzyme-deficient proteins of the invention. Substantially similar sequences share at least about 40%, more preferably at least about 60%, and most preferably at least about 80% sequence identity. Where the sequences diverge, the differences are generally point insertions/deletions or conservative substitutions, i.e. a cysteine/threonine or serine substitution, an acidic/acidic or hydrophobic/hydrophobic amino acid substitution, etc. Conservative substitution or mutations as used herein denotes the replacement of an amino acid residue by another, biologically similar residue. Examples of conservative substitutions include the substitution of one hydrophobic residue such as isoleucine, valine, leucine or methionine for another, or the substitution of one polar residue for another, such as the substitution of arginine for lysine, glutamic for aspartic acid, or glutamine for asparagine, and the like. Conservative substitution is also intended to include differential splicing and repeats of various sequences, such as those seen in the various isoforms described herein (e.g. those seen in human, murine and chick polypeptides/proteins). The term conservative substitution as used herein also includes the use of a substituted amino acid in place of an unsubstituted parent amino acid provided that described homolog having the substituted polypeptide also stimulates neurite outgrowth. The subject polypeptides/peptides are solated, meaning unaccompanied by at least some of the material with which they are associated in their natural state. Generally, an isolated polypeptide/peptide constitutes at least about 1%, preferably at least about 10%, and more preferably at least about 50% by weight of the total polypeptide/peptide in a given sample. By pure peptide/polypeptide is intended at least about 60%, preferably at least 80%, and more preferably at least about 90% by weight of total polypeptidelpeptide. Included in the subject polypeptide/peptide weight are any atoms, molecules, groups, etc. covalently coupled to the subject polypeptides/peptides, such as detectable labels, glycosylations, phosphorylations, etc. The subject polypeptides/peptides may be isolated or purified in a variety of ways known to those skilled in the art depending on what other components are present in the sample and to what, if anything, the polypeptide/peptide is covalently linked. Purification methods include electrophoretic, molecular, immunological and chromatographic techniques, especially affinity chromatography and RP-HPLC in the case of peptides. The subject polypeptides/peptides generally comprise naturally occurring amino acids but D-amino acids or amino acid mimetics coupled by peptide bonds or peptide bond mimetics may also be used. Amino acid mimetics are other than naturally occurring amino acids that conformationally mimic the amino acid for the purpose of the requisite polypeptid specificity. Suitable mimetics are known to those of ordinary skill in the art and include beta-gamma-delta amino and imino acids, cyclohexylalanine, adamantylacetic acid, etc., modifications of the amide nitrogen, the alpha-carbon, amide carbonyl, backbone modifications, etc.

The invention also relates to a recognition agent capable of recognizing an isolated nucleotide sequence of the invention or a polypeptide of the invention, whereby the agent is e.g. an antibody or an anti-sense-construct. In the context of the present invention the term recognition agent refers to molecules which interact with proteins or nucleic acid sequences encoding said proteins or fragments thereof. In a embodiment of the present invention the recognition agent is for instance a polyclonal or monoclonal antibody, a lectin, an oligonucleotid or an anti-sense construct. The said proteins, or fragments thereof, may be used to produce polyclonal or monoclonal antibodies, which also may serve as sensitive detection reagents for the presence and accumulation of polypeptides in cultured cells or tissues from living patients; the term patient refers to both humans and animals. The full-length proteins or fragments of the proteins may be used to advantage to generate an array of monoclonal antibodies specific for various epitopes of the proteins, thereby potentially providing even greater sensitivity for detection of the proteins in cells or tissues. Although the recognition agent will conveniently be an antibody, other recognition agents are known or may become available, and can be used in the present invention. For example, antigen binding domain fragments of antibodies, such as Fab fragments, can be used. Also, so-called RNA aptomers may be used. Therefore, unless the context specifically indicates otherwise, the term antibody as used herein is intended to include other recognition agents. Where antibodies are used, they may be polyclonal or monoclonal. Optionally, the antibody can produced by a method so that it recognizes a preselected epitope of said proteins. Polyclonal or monoclonal antibodies immunologically specific for the said proteins may be used in a variety of assays designed to localize and/or quantitate the proteins. Such assays include, but are not limited to: (1) flow cytometric analysis; (2) immunochemical localization of the protein in cultured cells or tissues; and (3) immunoblot analysis; e.g., dot blot, Western blot, of extracts from cells and tissues. Additionally, as described above, such antibodies can be used for the purification of said proteins; e.g, affinity column purification, immunoprecipitation.

Antibody refers in the context of the present invention to a polypeptide substantially encoded by an immunoglobulin gene or immunoglobulin genes, or fragments thereof, which specifically bind and recognize an analyte (antigen). Antibodies exist, e.g., as intact immunoglobulins or as a number of well characterized fragments produced by digestion with various peptidases. The term antibody, as used herein, also includes antibody fragments either produced by the modification of whole antibodies or those synthesized de novo using recombinant DNA methodologies. An antibody specifically binds to or is specifically immunoreactive with a protein when the antibody functions in a binding reaction which is determinative of the presence of the protein in the presence of a heterogeneous population of proteins and other biologics. Thus, under designated immunoassay conditions, the specified antibodies bind preferentially to a particular protein and do not bind in a significant amount to other proteins present in the sample. Specific binding to a protein under such conditions requires an antibody that is selected for its specificity for a particular protein. A variety of immunoassay formats may be used to select antibodies specifically immunoreactive with a particular protein. For example, solid-phase ELISA immunoassays are routinely used to select monoclonal antibodies specifically immunoreactive with a protein. The present invention also relates to use recognition agents for diagnosis, prophylactic or therapeutic treatment of degenerative nerve disease and neurogenerative damage.

The invention also provides a method for identifying a receptor (candidate molecule) that binds a polypeptide of the invention. The method includes the steps of: (a) providing a protein/polypeptide of the invention; (b) contacting the protein/polypeptide with the candidate molecule; and (c) detecting binding of the candidate molecule to the protein/polypeptide. The invention also relates to a kit for screening the candidate molecule that binds a nucleotide sequence according of the invention, a polypeptide of the invention and/or an recognition agent of the invention comprising a nucleotide sequence of the invention, a polypeptide according of the invention and/or an recognition agent of the invention. Receptors which interacts with a protein/polypeptide of the invention are e.g.: Eph receptors, semaphorins, trkA, trkB, trkC and the p75 neurotrophin receptor, NCAMs and related membrers of the IgG supefamily involved in axonal pathfinding, neuropilin, LPPs. Using the disclosed proteins or polypeptides/peptides, receptors are identified by a variety of techniques known to those skilled in the art where a ligand to the target receptor is known, including expression cloning. Generally, COS cells are transfected to express a fetal brain cDNA library or PCR product and cells producing polypeptides/peptides which bind a target polypeptide/peptide are isolated. Alternatively, PCR primers based upon sequences disclosed herein are used to amplify PCR product from such tissues/cells. Other receptor/ligand isolation methods using immobilized ligand or antibody are known to those skilled in the art. Additional peptides with receptor binding specificity are identified by a variety of ways including crosslinking to receptor or specific antibody, or preferably, by screening such peptides for binding or disruption of peptide-peptide receptor binding. For example, peptide routants, including deletion mutants are generated from and used to identify regions important for specific protein-ligand or protein-protein interactions, for example, by assaying for the ability to mediate axon outgrowth in cell-based assays as described herein. Further, structural x-ray crystallographic and/or NMR data of the disclosed protein are used to rationally design binding molecules of determined structure or complementarity for modulating axon outgrowth and guidance. Additional polypeptide/peptide/receptor-specific agents include specific antibodies that can be modified to a monovalent form, such as Fab, Fab′, or Fv, specifically binding oligopeptides or oligonucleotides and most preferably, small molecular weight organic receptor agonists. For example, the disclosed peptide and receptor peptides are used as immunogens to generate specific polyclonal or monoclonal antibodies. See, Harlow and Lane (1988) Antibodies, A Laboratory Manual, Cold Spring Harbor Laboratory, for general methods. Anti-idiotypic antibody, especially internal imaging anti-ids are also prepared using the disclosures herein.

Other prospective peptide/receptor specific agents are screened from large libraries of synthetic or natural compounds. For example, numerous means are available for random and directed synthesis of saccharide, peptide, and nucleic acid based compounds. Alternatively, libraries of natural compounds in the form of bacterial, fungal, plant and animal extracts are available or readily producible. Additionally, natural and synthetically produced libraries and compounds are readily modified through conventional chemical, physical, and biochemical means. Useful agents are identified with assays employing a compound comprising the subject polypeptides/peptides or encoding nucleic acids. A wide variety of in vitro, cell-free binding assays, especially assays for specific binding to immobilized compounds comprising peptide/receptor polypeptide/peptide find convenient use. See, e.g. Fodor et al (1991) Science 251, 767 for the light directed parallel synthesis method. Such assays are amenable to scale-up, high throughput usage suitable for volume drug screening. While less preferred, cell-based assays may be used to determine specific effects of prospective agents on e.g. polypeptide and/or receptor function.

The present invention also relates to a pharmaceutical composition comprising a therapeutically effective amount of the said nucleic acid sequence and/or the said polypeptide and one or more pharmaceutically acceptable adjuvant, excipient, carrier, buffer, diluent and/or customary pharmaceutical auxiliary. In a preferred embodiment of the invention the protein/polypeptide of the invention can be administered in a pharmaceutically acceptable formulation. The present invention pertains to any pharmaceutically acceptable formulations, such as synthetic or natural polymers in the form of macromolecular complexes, nanocapsules, microspheres, or beads, and lipid-based formulations including oil-in-water emulsions, micelles, mixed micelles, synthetic membrane vesicles, and resealed erythrocytes. In addition to the enzyme-deficient C3 botulinum protein species and the pharmaceutically acceptable polymer, the pharmaceutically acceptable formulation used in the method of the invention can comprise additional pharmaceutically acceptable carriers and/or excipients. As used herein, pharmaceutically acceptable carrier includes any and all solvents, dispersion media, coatings, antibacterial and anti fungal agents, isotonic and absorption delaying agents, and the like that are physiologically compatible. For example, the carrier can be suitable for injection into the cerebrospinal fluid. Excipients include pharmaceutically acceptable stabilizers and disintegrants. In another embodiment, the pharmaceutically acceptable formulations comprise lipid-based formulations. Any of the known lipid-based drug delivery systems can be used in the practice of the invention. For instance, multivesicular liposomes (MVL), multilamellar liposomes (also known as multilamellar vesicles or MLV), unilamellar liposomes, including small unilamellar liposomes (also known as unilamellar vesicles or SUV) and large unilamellar liposomes (also known as large unilamellar vesicles or LUV), can all be used so long as a sustained release rate of the encapsulated enzyme-deficient C3 botulinum protein species can be established. In one embodiment, the lipid-based formulation can be a multivesicular liposome system. The composition of the synthetic membrane vesicle is usually a combination of phospholipids, usually in combination with steroids, especially cholesterol. Other phospholipids or other lipids may also be used. Examples of lipids useful in synthetic membrane vesicle production include phosphatidylglycerols, phosphatidylcholines, phosphatidylserines, phosphatidylethanolamines, sphingolipids, cerebrosides, and gangliosides. Preferably phospholipids including egg phosphatidylcholine, dipalmitoylphosphatidylcholine, distearoylphosphatidylcholine, dioleoylphosphatidylcholine, dipalmitoylphosphatidylglycerol, and dioleoylphosphatidylglycerol are used. In another embodiment, the composition containing the neurite outgrowth-promoting polypeptide may be incorporated or impregnated into a bioabsorbable matrix. In addition, the matrix may be comprised of the said biopolymer. A suitable biopolymer for the present invention can include also one or more macromolecules selected from the group consisting of collagen, elastin, fibronectin, vitronectin, laminin, polyglycolic acid, hyaluronic acid, chondroitin sulfate, dermatan sulfate, heparin sulfate, heparin, fibrin, cellulose, gelatin, polylysine, echinonectin, entactin, thrombospondin, uvomorulin, biglycan, decorin, and dextran. The formulation of these macromolecules into a biopolymer is well known in the art. In constructing the matrix, it may be useful for the matrix to further include a substructure for purposes of administration and/or stability. Suitable substructures include freeze dried sponge, powders, films, flaked or broken films, aggregates, microspheres, fibers, fiber bundles, or a combination thereof. In addition, the matrix may be attached to a solid support for administration purposes. Suitable supports depend upon the specific use and can include a prosthetic device, a porous tissue culture insert, an implant, a suture, and the like. Therapeutic compositions of the present invention may include a physiologically tolerable carrier together with at least one species of neurite outgrowth-promoting polypeptide of this invention as described herein, dispersed therein as an active ingredient. It is further contemplated that the various polypeptides as described herein can be used therapeutically in a variety of applications. For example, as described above, a variety of useful compositions and formats, including bioabsorbable materials or matrices may be used in conjunction with the polypeptides of the present invention to coat the interior of tubes used to connect severed neurons; they may be added directly to suture materials or incorporated in bioabsorbable materials in and on sutures; further, they may be utilized on/in implants and prosthetic devices, either alone or in conjunction with other bioabsorbable and supporting materials. Thus in one embodiment, a pharmacologically active polypeptide of this invention can be incorporated into a bioabsorbable matrix, which matrix can be formulated into a variety of mediums, including a semi-solid gel, a liquid permeable but porous insoluble matrix, or a porous biopolymer as described further herein. A variety of useful compositions, including bioabsorbable materials (e.g., collagen qels) may be used in conjunction with a polypeptide of the present invention in a variety of therapeutic applications. For example, a neurite outgrowth-promoting polypeptide can be used to coat the interior of tubes used to connect severed neurons; they may be added directly to suture materials or incorporated in bioabsorbable materials in and on sutures; further, they may be utilized on/in implants, and prosthetic devices, either alone or in conjunction with other bioabsorbable and supporting materials. In a preferred embodiment, the therapeutic composition is not immunogenic when administered to a human patient for therapeutic purposes.

A therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, propylene glycol, polyethylene glycol and other solutes. Liquid compositions can also contain liquid phases in addition to and to the exclusion of water. Exemplary of such additional liquid phases are glycerin, vegetable oils such as cottonseed oil, organic esters such as ethyl oleate, and water-oil emulsions. A therapeutic composition contains a polypeptide of the present invention, typically an amount of at least 0.1 weight percent of polypeptide per weight of total therapeutic composition. A weight percent is a ratio by weight of polypeptide to total composition. Thus, for example, 0.1 weight percent is 0.1 grams of polypeptide per 100 grams of total composition.

A therapeutically effective amount of a neurite outgrowth-promoting polypeptide-containing composition, or beneficial compound therein, is a predetermined amount calculated to achieve the desired effect, i.e., to effectively promote neurite outgrowth of targeted neuronal cells. In addition, an effective amount can be measured by improvements in one or more symptoms occurring in a patient. Effective amounts can be measured by improvements in neuronal cell survival, axonal regrowth, and connectivity following axotomy using well known methods. Improvements in neuronal regeneration in the central nervous system (CNS) and peripheral nervous system (PNS) are also indicators of the effectiveness of treatment with the disclosed compounds and compositions, as are improvements in nerve fiber regeneration following traumatic lesions. Thus, the dosage ranges for the administration of a polypeptide of the invention are those large enough to produce the desired effect in which the condition to be treated is ameliorated. The dosage should not be so large as to cause adverse side effects. Generally, the dosage will vary with the age, condition, and sex of the patient, and the extent of the disease in the patient, and can be determined by one of skill in the art. The dosage can be adjusted by the individual physician in the event of any complication. The compositions are administered in a manner compatible with the dosage formulation, and in a therapeutically effective amount. A therapeutic amount of a polypeptide composition of this invention is an amount sufficient to produce the desired result, and can vary widely depending upon the disease condition and the potency of the therapeutic compound. The quantity to be administered depends on the subject to be treated, the capacity of the subject's system to utilize the active ingredient, and the degree of therapeutic effect desired. Precise amounts of active ingredient required to be administered depend on the judgment of the practitioner and are peculiar to each individual. However, suitable dosage ranges for systemic application are disclosed herein and depend on the conditions of administration.

Suitable regimes for administration are also variable, but are typified by an initial administration followed by repeated doses at one or more hour intervals by a subsequent administration. A therapeutically effective amount of a polypeptide of this invention is typically an amount such that when it is administered in a physiologically tolerable composition, it is sufficient to achieve a plasma or local concentration of from about 0.1 to 1,000 micromolar (uM), preferably about 1 to 100 uM. Alternatively, the dosage can be metered in terms of the body weight of the patient to be treated. In this case, a typical dosage of a therapeutic composition is formulated to deliver a pharmacologically active polypeptide of this invention is amount of about 0.1 microgram (ug) to 100 ug per kilogram (kg) body weight, or more preferably about 1 to 50 ug/kg. Furthermore, certain utilities of the present invention involve local administration of a pharmacologically active polypeptide to a site of lesion, and therefore is best expressed in unit dosage form. Such local administration is typically by topical or local administration of a liquid or gel composition containing about 1 to 1000 micrograms (ug) of active polypeptide per milliliter (ml) of composition, preferably about 5 to 500 ug/ml, and more preferably about 10 to 100 ug/ml. Thus a therapeutic composition can be administered via a solid, semi-solid (gel) or liquid composition, each providing particular advantages for the route of administration. A polypeptide of the invention can be administered parenterally by injection or by gradual infusion over time. For example, a polypeptide of the invention can be administered topically, locally, perilesionally, perineuronally, intracranially, intravenously, intrathecally, intramuscularly, subcutaneously, intracavity, transdermally, dermally, or via an implanted device, and they may also be delivered by peristaltic means. In general, local, prerilesional, intrathecal, perineuronal, or intra-CNS administration is preferred. The therapeutic compositions containing a polypeptide of this invention are conventionally administered intravenously, as by injection of a unit dose, for example. The term unit dose when used in reference to a therapeutic composition of the present invention refers to physically discrete units suitable as unitary dosage for the subject, each unit containing a predetermined quantity of active material calculated to produce the desired therapeutic effect in association with the required diluent; i.e., carrier, or vehicle. Alternatively, continuous intravenous infusion sufficient to maintain therapeutically effective concentrations in the blood are contemplated. Therapeutically effective blood concentrations of a polypeptide of the present invention are in the range of about 0.01 uM to about 100 uM, preferably about 1 uM to about 10 uM. The terms therapeutically effective or effective, as used herein, may be used interchangeably and refer to an amount of a therapeutic composition of the present invention—e.g., one containing a neurite outgrowth-promoting polypeptide of this invention. For example, a therapeutically effective amount of a neurite outgrowth-promoting polypeptide-containing composition, or beneficial compound therein, is a predetermined amount calculated to achieve the desired effect, i.e., to effectively promote neurite outgrowth of neurons in an individual to whom the composition is administered.

The polypeptides of the present invention are typically administered as a pharmaceutical composition in the form of a solution, gel or suspension. However, therapeutic compositions of the present invention may also be formulated for therapeutic administration as a tablet, pill, capsule, aerosol, sustained release formulation or powder.

In another embodiment of the method of the invention, the pharmaceutically acceptable formulation provides sustained delivery, e.g., slow release of the protein/peptide of the invention to a subject for at least one, two, three, or four weeks after the pharmaceutically acceptable formulation is administered to the subject. As used herein, the term subject is intended to include animals susceptible to CNS/PNS injuries, preferably mammals, most preferably humans. In a preferred embodiment, the subject is a primate. In an even more preferred embodiment, the primate is a human. Other examples of subjects include dogs, cats, goats, and cows. As used herein, the term sustained delivery is intended to include continual delivery of enzyme-deficient C3 botulinum protein species in vivo over a period of time following administration, preferably at least several days, a week or several weeks. In one embodiment, the pharmaceutically acceptable formulation provides sustained delivery of the enzyme-deficient C3 botulinum protein species to a subject for less than 10-30 days after the enzyme-deficient C3 botulinum protein species is/are administered to the subject. For example, the pharmaceutically acceptable formulation, e.g., slow release formulation, can provide sustained delivery of the enzyme-deficient C3 botulinum protein species to a subject for one, two, three or four weeks after the enzyme-deficient C3 botulinum protein species is administered to the subject. Alternatively, the pharmaceutically acceptable formulation may provide sustained delivery of the enzyme-deficient C3 botulinum protein species to a subject for more than 10-30 days after enzyme-deficient C3 botulinum protein species is/are administered to the subject. The pharmaceutical formulation, used in the method of the invention, contains a therapeutically effective amount of enzyme-deficient C3 botulinum protein species. A therapeutically effective amount of slow release formulation refers to an amount effective, at dosages and for periods of time necessary, to achieve the desired result. A therapeutically effective amount of slow release formulation may vary according to factors such as the disease state, age, and weight of the subject, and the ability of the enzyme-deficient C3 botulinum protein species to elicit a desired response in the subject. Dosage regimens may be adjusted to provide the optimum therapeutic response. The invention in another embodiment, provides a pharmaceutical composition consisting essentially of a enzyme-deficient C3 botulinum protein species and a pharmaceutically acceptable carrier and methods of use thereof to modulate axonal outgrowth by contacting neurons with the slow release formulation or composition. By the term consisting essentially of is meant that the pharmaceutical composition does not contain any other modulators of neuronal growth such as, for example, nerve growth factor (NGF), brain derived neurotrophic factor (BDNF), ciliary neurotrophic factor (CNTF), glial derived neurotrophic factor (GDNF). In one embodiment, the slow release composition of the invention can be provided as a packaged formulation. The packaged slow release formulation may include a pharmaceutical composition of the invention in a container and printed instructions for administration of the composition for treating a subject having a disorder associated with an injury of central nervous system neurons, e.g., an injury to retinal ganglion cells, a spinal cord injury or a traumatic brain injury. In vitro treatment of neurons with the composition: neurons can further be contacted with a therapeutically effective amount of a enzyme-deficient C3 botulinum protein species in slow release composition in vitro. Accordingly, neurons or neuronal cells can be isolated from a subject and grown in vitro, using techniques well known in the art. Briefly, a neuronal cell culture can be obtained by allowing neuron cells (see above) to migrate out of fragments of neural tissue adhering to a suitable substrate (e.g., a culture dish) or by disaggregating the tissue, e.g., mechanically or enzymatically, to produce a suspension of neuronal cells. For example, the enzymes trypsin, collagenase, elastase hyaluronidase, Dnase, pronase, dispase, or various combinations thereof can be used. Trypsin and pronase give the most complete disaggregation but may damage the cells. Collagenase and dispase give a less complete dissagregation but are less harmful. Such cells can be subsequently contacted with a enzyme-deficient C3 botulinum protein species at levels and for a duration of time as described above. Once modulation of axonal outgrowth has been started in the neuron, these primed cells can be re-administered to the subject. According to the present invention, a pharmaceutical composition that is highly safe and has excellent neurite extending effect on cells can be provided, and therefore, a method for extending neurites and a method for preventing and/or treating neurodegeneration diseases are provided. In particular, it is effective to use a composition containing enzyme-deficient, not Rho-inactivating, C3 botulinum protein species as an active ingredient of said slow release composition. The composition for extending neurites of the present invention can be used as a pharmaceutical, a quasi-drug or a food—only if digestion is prevented and uptake via the epithelial cells of the gut is guaranteed—, and are effective to extend neurites and to prevent and/or treat neurodegeneration diseases such as Alzheimer's dementia and encephalic ischemia.

The invention further contemplates a neurite outgrowth-promoting apparatus that comprises a bioabsorbable matrix and an effective amount of a pharmacologically active agent capable of inducing neurite outgrowth, wherein the agent comprises a polypeptide that promotes neurite outgrowth as described herein. The matrix can be in the form of a solid support and the pharmacologically active agent can be attached to the substrate. The agent can optionally be incorporated into the bioabsorbable matrix, which can be comprised of a biopolymer of a variety of materials. The matrix can further include a substructure comprising freeze dried sponge, powders, films, flaked or broken films, aggregates, microspheres, fibers, fiber bundles, or a combination, thereof. The solid support can be formulated into a prosthetic device, a porous tissue culture insert, an implant and a suture. The matrix can be adapted for use in tissue culture. The invention also contemplates a method of promoting neurite outgrowth in a subject which comprises administering to the subject a physiologically tolerable composition containing a therapeutically effective amount of a neurite outgrowth-promoting polypeptide as described herein. The polypeptide can be incorporated into a bioabsorbable matrix, as described above for the apparatus of the invention. The invention contemplates a variety of apparati for use in practicing the methods of the invention, both in vitro and in vivo. As described in above for practicing the methods, the subject polypeptide can be incorporated into a bioabsorbable matrix which is formulated in a variety of solid and semi-solid formats which can comprise a apparatus for administering the polypepitde. Thus, the invention contemplates a neurite outgrowth-promoting apparatus that comprises a bioabsorbable matrix combined with an effective amount of a pharmacologically active agent capable of inducing neurite outgrowth of neuronal cells. The agent is a composition containing any one or more of the subject polypeptides of this invention in an amount effective to induce neurite outgrowth as defined herein. The apparatus can be formulated in a variety of configurations for adminstration purposes as described herein for the methods of treatment, and include combining the matrix with a solid support into a prosthetic device, a porous tissue culture insert, an implant, a suture, an entubation apparatus and the like. Solid supports (also described as solid surfaces or solid substrates) useful according to the present invention include supports made of glass, plastic, nitrocellulose, cross-linked dextrans, agarose in its derivatized and/or cross-linked form, polyvinyl chloride, polystyrene, cross-linked polyacrylamide, nitrocellulose- or nylon-based webs such as sheets, strips or paddles, tubes, plates, the wells of a microtiter plate such as those made from polystyrene or polyvinylchloride, and the like, and may take the form of a planar surface or microspheres to name a few variations. Useful solid support materials in this regard include the derivatized cross-linked dextran, agarose in its derivatized and/or cross-linked form, polystyrene beads about 1 micron to about 5 millimeters in diameter, polyvinyl chloride, polystyrene, cross-linked polyacrylamide, nitrocellulose- or nylon-based webs such as sheets, strips or paddles, tubes, plates, the wells of a microtiter plate such as those made from polystyrene or polyvinylchloride, and the like. In another embodiment, the invention discloses a method of preparing substrates (solid support) for the attachment of cells thereto useful for promoting neurite outgrowth, comprising providing a composition containing a polypeptide exhibiting neurite outgrowth-promoting activity of this invention and treating by coating or impregnating a matrix in or on the solid substrate with said polypeptide-containing composition. In various disclosed embodiments, the solid support or substrate may comprise glass, agarose, a synthetic resin material (e.g., nitrocellulose, polyester, polyethylene, and the like), long-chain polysaccharides, and other similar substances. The solid support can be formulated, as described herein, in a variety of administration formats for both in vitro or in vivo use, and the specific format need not be considered as limiting to the invention.

Therefore the present invention correspondingly provides a method for modulating neurite outgrowth of central or peripheral nervous system neurons in vitro or in vivo comprising contacting the neurons with the pharmaceutical composition such that neurite outgrowth is modulated. The composition for cell treatment of the present invention contains e.g. a physiologically acceptable carrier. Any physiologically acceptable carrier can be used as long as they are generally used to culture and grow cells, such as a culture medium. The content of the proteins/polypeptides of invention in the composition for cell treatment can be determined as appropriate by those skilled in the art. The composition for cell treatment can be produced by a method well known to those skilled in the art. The present invention provides also methods for modulating the neuronal or axonal outgrowth of central nervous system neurons.

The invention is based, at least in part, on the discovery that enzyme-deficient—not Rho-inactivating—C3 botulinum protein species and analogs or peptides thereof induce stimulation of neuronal or axonal outgrowth from mice neurons without effects on glial cells. Accordingly, the methods of the invention for modulating neuronal outgrowth of central or peripheral nervous system or neurons generally involve contacting the neurons with a protein or polypetide of the invention such that axonal or neuronal outgrowth is modulated. As used herein, the language modulating the axonal or neuronal outgrowth of central nervous system neurons is intended to include the capacity to stimulate axonal outgrowth of nervous system neurons to various levels, e.g., to levels which allow for the treatment of targeted CNS or peripheral nervous system (PNS) injuries. As used herein, the term outgrowth refers to the process by which processes or neurites grow out of a neuron. The outgrowth can result in a totally new axon or the repair of a partially damaged axon. Outgrowth is typically evidenced by extension of an neuronal process of at least 2-5 cell diameters in length. As used herein, the term CNS neurons is intended to include the neurons of the brain and the spinal cord, the term peripheral neuron (PNS neuron) includes neurons of spinal ganglia and ganglia of the vegetative nervous system. The term is not intended to include support or protection cells such as glial cells like astrocytes, microglia and the like. As used herein, the language contacting is intended to include both in vivo or in vitro methods of bringing an enzyme-deficient C3 botulinum protein species into proximity with a CNS neuron, such that the enzyme-deficient C3 botulinum protein species can modulate the outgrowth of neuronal or axonal processes from said neuron. The invention also provides methods for stimulating the outgrowth of central nervous system neurons following an injury. The method involves administering to a subject a enzyme-deficient C3 botulinum protein species. As used herein, the term injury is intended to include a damage which directly or indirectly affects the normal functioning of the CNS or PNS. For example, the injury can be damage to retinal ganglion cells; a traumatic brain injury; a stroke related injury; a cerebral aneurism related injury; a spinal cord injury, including monoplegia, diplegia, paraplegia, hemiplegia and quadriplegia; a neuroproliferative disorder or neuropathic pain syndrome. As used herein, the term stroke is art recognized and is intended to include sudden diminution or loss of consciousness, sensation, and voluntary motion caused by rapture or obstruction (e.g. by a blood clot) of an artery of the brain. As used herein, the term Traumatic Brain Injury is art recognized and is intended to include the condition in which, a traumatic blow to the head causes damage to the brain, often without penetrating the skull. Usually, the initial trauma can result in expanding hematoma, subarachnoid hemorrhage, cerebral edema, raised intracranial pressure, and cerebral hypoxia, which can, in turn, lead to severe secondary events due to low cerebral blood flow.

In a preferred method a damage is also due to infarction, traumatic injury, surgical lesion or a degenerative disorder of the central nervous system; preferred the damage has occurred to the spinal cord. Spinal cord function requires electrical conduction from one nerve cell to another through the extended axonal processes of these cells. After injury to the adult human spinal cord, these connections are interrupted, and the surviving nerve cells cannot communicate with one another to provide muscle control and sensation. Previous studies have indicated that the nerve cells are capable of reextending their axons if given an appropriate environment. Unfortunately, the adult spinal cord is an inappropriate environment because inhibitory molecules are expressed by non-neuronal supporting cells. The invention provides a method for treating a central nervous system disease, disorder or injury. The method includes administering to a mammal, e.g., a human, an effective amount of a protein/polypeptide of the invention. Exemplary diseases, disorders and injuries that may be treated using molecules and methods of the invention include, but are not limited to, cerebral injury, spinal cord injury, stroke, demyelinating diseases, e.g., multiple sclerosis, monophasic demyelination, encephalomyelitis, multifocal leukoencephalopathy, panencephalitis, Marchiafava-Bignami disease, Spongy degeneration, Alexander's disease, Canavan's disease, metachromatic leukodystrophy and Krabbe's disease. In a preferred method the degenerative disorder is selected from the group consisting of Parkinson's disease, amyotrophic lateral sclerosis, Alzheimer's disease, diffuse cerebral cortical atrophy, Lewy-body dementia, Pick disease, mesolimbocortical dementia, thalamic degeneration, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, Hallervorden-Spatz disease, Meige syndrome, familial tremors, Gilles de la Tourette syndrome, acanthocytic chorea, Friedreich ataxia, Holmes familial cortical cerebellar atrophy, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, and ophthalmoplegia.

In one embodiment protein/composition of the invention is administered by introduction into the central nervous system of the subject, e.g., into the cerebrospinal fluid of the subject. For therapeutic uses, the compositions and agents disclosed herein may be administered by any convenient way. Small organics are preferably administered orally; other compositions and agents are preferably administered parenterally, conveniently in a pharmaceutically or physiologically acceptable carrier, e.g., phosphate buffered saline, or the like. Typically, the compositions are added to a retained physiological fluid such as blood or synovial fluid. For CNS administration, a variety of techniques are available for promoting transfer of the therapeutic across the blood brain barrier including disruption by surgery or injection, drugs which transiently open adhesion contact between CNS vasculature endothelial cells, and compounds which facilitate translocation through such cells. As examples, many of the disclosed therapeutics are amenable to direct injection or infusion, topical, intratracheal/nasal administration e.g. through aerosal, intraocularly, or within/on implants e.g. fibers e.g. collagen, osmotic pumps, grafts comprising appropriately transformed cells, etc. In certain aspects of the invention, the protein/peptide/composition is introduced intrathecally, e.g., into a cerebral ventricle, the lumbar area, or the cistema magna. The pharmaceutically acceptable formulations can easily be suspended in aqueous vehicles and introduced through conventional hypodermic needles or using infusion pumps. Prior to introduction, the formulations can be sterilized with, preferably, gamma radiation or electron beam sterilization.

The invention relates to the use of a nucleotide sequence of the invention, a polypeptide of the invention and/or an recognition agent of the invention to promote neural growth without undisered impairing glial proliferation

The invention provides the use of a nucleotide sequence of the invention, a polypeptide of the invention and/or an recognition agent of the invention for the manufacture of an agent for diagnosis, prophylactic and/or therapeutic treatment of a damage of the central nervous system. The invention relates also to the use of a nucleotide sequence of the invention, a polypeptide of the invention and/or an recognition agent of the invention to induce a expansion and/or differentiation of stem cells; e.g. neuronal stem cells. These include embryonic as well as adult stem cells which can be grown and differentiated in vitro before implantation to rescue certain neuronal functions. In this case the C3-bot proteins or peptides can be applied in such a way that neuronal differentiation has been just initiated. After implantation full axonal growth with these “C3-bot-primed” neurons may be obtained.

The polypeptides, proteins and compositions of the invention are useful: the discovery that regions of the enzyme-deficient, not directly Rho-inactivating, C3 botulinum polypeptides/proteins described herein can promote neurite outgrowth, and the accompanying identification of pharmacologically active polypeptides, provides agents for use in improving nerve regeneration or promoting nerve survival, in treating peripheral nerve injury and spinal cord injury, and in stimulation of growth of endogenous, implanted or transplanted CNS tissue. The present invention therefore also provides a method of promoting regeneration of an injured or severed nerve or nerve tissue, or promoting neurite outgrowth in neuronal cells under a variety of neurological conditions requiring neuronal cell outgrowth. The method comprises contacting a neuronal cell capable of extending neurites, or an injured or severed nerve, with a cell culture system comprising a substrate containing a neurite outgrowth-promoting polypeptide of this invention in an amount effective to promote neurite outgrowth. The method may be carried out in vitro or in vivo. Any of a variety of mammalian neuronal cells can be treated by the present method in the cell culture system, including neuronal cells from brain, CNS, peripheral nerves and the like. In addition, the cells can be from any of a variety of mammalian species, including human, mouse, chicken, and any other mammalian species, including the agricultural stock and non-domesticated mammals. The neurite outgrowth-promoting composition can be attached to the substrate, can be contacted in the liquid phase or in a collagen gel phase. The composition may contain the subject polypeptide in the form of a fusion protein as described herein. The method may be practiced using the subject polypeptide in any of the various apparati format described herein. The methods can optionally be practiced in combination with contacting the neuronal cells or nerves with other agents capable of promoting neuron survivals growth, differentiation or regeneration.

In vitro: In a preferred embodiment, the invention contemplates in vitro methods and kits for culturing neuronal cells under conditions where the subject polypeptides are used to promote neurite outgrowth, and can include methods for detecting the presence and amount of stimulation of neurite outgrowth in the cultured neuronal cells. Various proteins and polypeptides disclosed herein are useful according to the within-disclosed methods. Appropriate cells are prepared for use in a neurite outgrowth assay. For example, a preparation of cortical neuron-cells is described in the Examples. The proteins and polypeptides of the present invention are therefore useful in a variety of applications relating to cell and tissue cultures. For example, in one embodiment, a method of promoting neurite outgrowth of neuronal cells in a cell culture system comprises the steps of (1) introducing neuronal cells into tissue culturing conditions comprising a culture medium; and (2) introducing a polypeptide of the present invention having neurite outgrowth-promoting activity into the culture medium in an amount effective to promote neurite outgrowth stimulating conditions in the culture. In another embodiment, a method of promoting neurite outgrowth of neuronal cells in a cell culture system comprises the steps of (1) immobilizing on the substrate a polypeptide of the present invention having neurite outgrowth-promoting activity; and (2) contacting neuronal cells with the substrate under tissue culturing conditions.

In vivo: The various proteins and polypeptides disclosed herein are also useful in a variety of therapeutic applications as described herein. The present therapeutic methods are useful in treating peripheral nerve damage associated with physical or surgical trauma, infarction, bacterial or viral infection, toxin exposure, degenerative disease, malignant disease that affects peripheral or central neurons, or in surgical or transplantation methods in which new neuronal cells from brain, spinal cord or dorsal root ganglia are introduced and require stimulation of neurite outgrowth from the implant and innervation into the recipient tissue. Such diseases further include but are not limited to CNS/PNS lesions, gliosis, Parkinson's disease, Alzheimer's disease, neuronal degeneration, and the like. The present methods are also useful for treating any disorder which induces a gliotic response or inflammation. In treating nerve injury, contacting a therapeutic composition of this invention with the injured nerve soon after injury is particularly important for accelerating the rate and extent of recovery. Thus the invention contemplates a method of promoting neurite outgrowth in a subject, or in selected tissues thereof, comprising administering to the subject or the tissue a physiologically tolerable composition containing a therapeutically effective amount of a neurite outgrowth-promoting polypeptide of the present invention. In one embodiment, a severed or damaged nerve may be repaired or regenerated by surgically entubating the nerve in an entubalation device in which an effective amount of a neurite outgrowth-promoting polypeptide of this invention can be applied to the nerve. In a related embodiment, a polypeptide of the invention can be impregnated into an implantable delivery device such as a cellulose bridge, suture, sling prosthesis or related delivery apparatus. The composition containing the neurite outgrowth-promoting polypeptide may be incorporated or impregnated into a bioabsorbable matrix, with the matrix being administered in the form of a suspension of matrix, a gel or a solid support.

These and other advantages of the present invention will become apparent to those skilled in the art upon reading and understanding the following examples.

The following examples are intended to illustrate but not limit the invention. While they are typical of those that might be used, other procedures known to those skilled in the art may alternatively be used.

### EXAMPLES

Recombinant C3 Toxins and C3-GFP cDNA Vectors

Recombinant proteins: The gene of C. botulinum C3 (accession No. X59039) (Popoff et al. 1991), or mutants of C3 harbouring glutamine (Q) or alanine (A) instead of glutamate (E) in position 174 (C3-botE174A (SEQ ID NO: 2), C3-botE174Q (SEQ ID NO: 1)) and the gene of C. limosum transferase (accession No. X872155) (Böhmer et al. 1996), were cloned into pGEX expression vector. After expression in E. coli, GST-C3 fusion proteins were affinity purified on glutathion-Sepharose followed by thrombin cleavage to obtain C3 proteins. The gene of S. aureus transferase strain HMI (accession No AJ277173) (Wilde et al., 2001a) was cloned into pQE30. After expression in E. coli His-tagged transferase were purified on nickel-columns.

C3-bot used in our study is documented under at least two different accession numbers (accession No. x59039=CM41767 (Popoff, M. R., Hauser, D., Boquet, P., Eklund, M. W. and Gill, D. M. (1991): Characterization of the C3 gene of Clostridium botulinum types C and D and its expression in Escherichia coli. Infect. Immun. 59: 3673-3679). There exist two additional isoforms derived also from C. botulinum which differ among each other by only few amino acids (Acc# Q00901; Nemoto, Y., Namba, T., Kozaki, S. and Narumiya, S. (1991): Clostridium botulinum C3 ADP-ribosyltransferase gene. Cloning, sequencing, and expression of a functional protein in Escherichia coli. J. Biol. Chem. 266, 19312-19319 and Acc# A41021; Moriishi, K., Syuto, B., Yokosawa, N., Oguma., K., Saito, M.: Purification and characterization of ADP-ribosyltransferases (exoenzyme C3) of Clostridium botulinum type C and D strains. J. Bacteriol. 173, 6025-6029 (1991)). These two C3 isoforms resembles our C3 bot (accession No. x59039=CM41767) more than C3 limosum (which has no neurotrophic effect); other C3bot isoforms (Acc# Q00901 and Acc#A41021) also possess neurotrophic effects.

Vectors: The gene of C. botulinum C3 (accession No. X590399), the mutant forms C3-botE174A and C3-botE174Q and C3-stau2 and the mutant C3-stau2E174Q were cloned into pEGFP eukaryotic expression vector.

Primary Culture of Mouse Hippocampal Neurons

Hippocampal or cortical neurons were prepared from 17 or 15 day old fetal NMRI mice, respectively. Dissected pieces of hippocampi or cortices were rinsed twice with PBS, then with dissociation medium (MEM supplemented with 10% fetal calf serum, 100 IE insuline/l, 0.5 mM glutamine, 100 U/ml penicillin/streptomycin, 44 mM glucose and 10 mM HEPES buffer) and dissociated mechanically. The suspension was centrifuged at 210×g for 2 min at 21° C., redissociated in starter medium (serum free neurobasal medium supplemented with B27, 0.5 mM glutamine, 100 U/ml penicillin/streptomycin and 25 μM glutamate) and plated on cover slips precoated with 0.5% poly-L-lysine dissolved in PBS layered in 24 er multiwells at a density of 20,000 cells/well. All ingredients were obtained from Gibco/BRL Life Technologies, Eggenstein, Germany. Neurons were cultivated up to 21 days in vitro (DIV) in an humidified atmosphere with 10% CO2.

One day after plating neurons, the various C3 derivatives were added to the culture medium at the indicated concentrations, usually 20 nM. 5 days later neurons were fixed with 4% formaldehyde dissolved in 0.1 M phosphate buffer pH 7.4. Fixed cells were treated with phosphate buffered saline (PBS) for 15 min and subsequently permeabilised for 30 min at room temperature (RT) using 0.3% Triton X-100 dissolved in PBS. Neurons or astrozytes were stained by antibodies against neurofilament protein (200 kDa) or glial fibrillary acidic protein (GFAP), respectively and immunoreacativity was visualized using anti-mouse IgG coupled to Cy2 (Jackson Immuno Research Laboratories, West Grove, Pa.).

Photomicrographs were taken from individual cells (neurons or astrozytes) and length and number of branches or processes were analysed morphometrically.

In some experiments hippocampal primary cultures were transfected using the cDNA vectors indicated. Transfected cells visualized by the green fluorescence protein were morphometrically analysed as given above.

Transfection of neurons was performed with 0.3 μg cDNA/15 mm dish using the effectene system (Life Technology) according to the manufacturers description. Transfected neurons were fixed after 24 h and subjected to morphometric analysis.

ADP Ribosylation Reaction

Hippocampal primary cultures were treated with the various C3 constructs for 5 days. Then the medium was removed and cells were frozen at −80° C. The frozen cultures were scraped and homogenized followed by centrifugation (10 min×2000 g) to prepare the post-nuclear supernatant, which was subjected to in vitro ADP-ribosylation using C3-bot toxin. To this end, 25 μg of hippocampal lysate proteins were incubated in the presence of 50 mM HEPES buffer pH 7.4 supplemented with 2 mM MgCl2, 1 mM dithiotheitol, 50 mM NaCl and 0.3 μM [P-32]NAD at 37° C. for 15 min. Thereafter, Laemmli sample buffer was added, boiled for 10 min at 95° C. and subjected to 12.5% SDS-PAGE. After staining, de-staining and drying of the gel, radioactivity was analysed by phosphorimager.

Rho-Independent Effect of C3 Proteins from Clostridium botulinum

The invention arises from the discovery that C3 proteins derived from Clostridium botulinum exert neurotrophic activity in nanomolar concentrations without interfering with Rho. Using C3-proteins with or without ADP-ribosyltransferase activity, one has to consider the overall function of Rho proteins for neuronal development and especially neurite formation and growth. Rho has been implicated in regulation of diverse cellular functions including neuronal growth cone formation and axon as well as dendrite elongation. Using a variety of neuronal preparations Rho has been suggested to either inhibit neurite formation (Jin and Strittmatter, 1997; Lehmann et al., 1999; Ymashita et al., 1999; Bito et a., 2000; Dergham et al., 2002) or to promote axonal and dendritic growth (Threadgill et al., 1997). Most of these studies have relied on transfection with dominant mutant forms of Rho or mechanical trituration using C3 transferases, but in μmolar concentrations. Generally it is currently believed that axonal as well as dendritic growth benefits from C3 treatment. The rational for this is the notion that Rho negatively regulates axonal growth and that inhibition of Rho by C3-catalysed ADP-ribosylation antagonises this negative impact. C3 protein from Clostridium botulinum (C3-bot) promotes axon growth when applied at nanomolar concentrations (FIG. 1). Most notably, the recombinant C3-bot derivatives devoid of ADP-ribosyltransferase activity generated by an exchange of the catalytic amino acid glutamate with either glutamine (rC3botE174Q) or alanine (rC3-botE174A) have the same axon promoting effects (FIGS. 2 and 3). These C3 proteins lack ADP-ribosyltransferase activity and they do not ADP-ribosylate interneuronal Rho even not after 5 days of incubation (FIG. 4). Thus, their neurotrophic effects are definitely not mediated by a direct inhibition of the Rho protein (see FIG. 4). Even more, axon promoting effects were not shared when using the related recombinant transferases from either C. limosum (rec. C3-lim) or Staphylococcus aureus (rec. C3-stau2) (FIGS. 2 and 3). The specificity of C3-bot is further underlined by the fact, that other C3 isoforms with functional ADP-ribosyltransferase activity exerted opposing effects on axonal growth and branching. Thus, the axon-promoting effects of C3-bot appear to be dominant over its effects on inactivating Rho when comparing C3-bot with C-3lim and C3-stau2 (FIGS. 3 and 4). In addition, the intracellular expression of C3 but not its enzyme-deficient isoforms reduces axon length and branching (FIG. 5) as it was also described for the dendritic growth in cortical neurons (Threadgill et al., 1997). Taken together rC3botE174A and rC3botE174Q promote axonal growth and branching without inactivating Rho by ADP-ribosylation.

Specificity of the Neurotrophic Effects

A further problem of the studies using C3transferases applied by trituration or extracellular application in the μmolar range is that indirect effects mediated by glial cells cannot be excluded. So far, the effects of glial cells on neuronal Rho by for example releasing growth factors like the glial-derived neurotrophic factor (GDNF), is not clear. GDNF and related factors (GDNF-family ligands) act via RET tyrosine kinase receptors thereby sustaining the development and maintenance of distinct sets of central and peripheral neurons (Airaksinen and Saarma, 2002). Another glial-mediated indirect neurotrophic effect involves the activity-dependent neurotrophic factor (ADNF), which is released by astrocytes. ADNF release in turn is mediated by the vasoactive intestinal polypeptide (VIP). VIP synthesis is upregulated in subsets of neurons during changes of the neuronal environment and acts as a neuroprotective neuropeptide (Gressens, 1999). In order to clearly distinguish between neurons and glial cells we performed a morphometric analysis of astrocytes in our culture system visualized by the glial acid fibrillary protein (GFAP). While rec. C3-bot induced a dramatic inrease in the length and number of processes in astrozytes, a process also referred to as stellation, this activity was not shared by the enzyme-deficient proteins rec. C3botE174A and rec. C3botE174Q. rec. C3-lim appeared to elicit similar effects, however in the presence of higher concentrations (FIGS. 6 and 7). Taken together the effects of rC3botE174A and rC3botE174Q are specific neurotrophic and not mediated or obscured by effects on astrozytes present in all nervous tissues.

Receptor Targets Responsible for Neurotrophic Effects of Rec C3-Bot Proteins and Enzymatically Deficicient Isoforms or Peptides of the Invention

Proteins and peptides of the invention have neurotrophic effects which can be elicited by nanomolar concentrations and do not require ADP-ribosyltransferase activity. So the axon promoting effects are mediated by extracellular structures. C3-bot proteins or one of the C3-bot-derived peptides of the invention exert their neurotrophic effects by one of the neurotrophic factor receptors or other transmembrane proteins. The structures included the receptor tyrosine kinases trkA, trkB and trkC and the p75 neurotrophin receptor. Comparable to neurotrophins C3-bot proteins activate directly receptor kinases thus promoting neurotrophic effects. Alternatively C3-bot proteins or peptides can act as receptor antagonist at the p75 neurotrophin receptor thereby exerting its neurotrophic effects (for review see Lee et al., 2001). The actions may be different at the axonal or dendritic compartment depending on other environmental requirements of the developing neuron. It can not be excluded that Rho may be indirectly involved in these processes but as stated above Rho is not required for axonal growth. As a third possibility C3-bot proteins enhances the neurotrophic effects of the various neurotrophic factors at their tyrosine kinase receptors.

Besides the neurotrophins and their trk-family receptors the Eph receptors (acitvated by ephrins A and B) represent the largest family of receptor kinases known so far. These receptors are mainly involved in the axon guidance pushing axons in a certain direction some also exhibiting repulsive effects. They are expressed complementary thereby separating pathes of outgrowing axons. These features are especially required to build up complex neuronal network where each axons has to find precisely its target. This also emplies that at early stages of neuronal development or during repair processes the time window for axonal growth is effectively used. C3-bot proteins or peptides can act via one of these Eph receptors (EphA1-EphA8 or EphB1-EphB6). As a first step an increased axonal growth as observed after C3-bot proteins may be advantageous to reach a distant target (Flanagan and Vanderhaeghen, 1998). The semaphorins via their surface receptors neuropilin 1 and 2 stop or even repulse growing axons. They act as physiological antagonists to the ephrins. C3-bot proteins may therefore block the semaphorin receptors neurophilin 1 and 2, thereby enhancing the neurotrophic effects of other neurotrophins.

Other possibilities for interference of C3-bot proteins and peptides with surface molecules of the neuronal/axonal plasma membrane include, the heptahelical Edg-receptors coupled to the heterotrimeric G-proteins G12/13 that mediate the various effects of LPA. Lysophospholipids like LPA represent ubiquitously present molecules the knowledge of their effects during neuronal development just has started to be elucidated. In vitro studies showed that LPA decreases neurite growth and also promotes growth cone collapse. LPA can act in an autocrine as well as in a paracrine fashion and may be released by surrounding tissue under physiological or pathophysiological conditions (Fukishima et al., 2000). So C3-bot proteins and peptides may act as Edg-receptor antagonists which by inhibiting the G12/13 mediated pathway enhances axonal growth. C3-bot proteins and peptides may directly be involved in the degradation of LPA by interfering with lipid phosphate phosphohydrolyses, integral membrane proteins present in the axonal/and or dendritic membrane which represent ectophosphorhydrolases.

Labelled C3-Bot Proteins or Peptides as Diagnostic Tools

Once the receptor(s) for C3-bot has been identified Alexa-labelled C3-bot proteins or peptides can be used to analyse changes in these receptors in biopsy material. Once the receptor(s) for C3-bot has been identified labelled C3-bot proteins or peptides can be used to analyse changes in these receptors in biopsy material.

To study the neuronal and the glial cell receptor for C3-bot we constructed a mutant C3-bot, accessible for fluorescence labelling. To this end, two cysteine residues were inserted by genetic techniques into C3-bot^{E174Q} to create C3-bot A1C-E174Q-K211C. (According to the published crystal structure, both the N-terminal as well as the C-terminal part of C3-bot are free and not interacting with the core protein of C3.) This mutant C3-bot was treated with the fluorescence dye Alexa-488 (Molecular Probes) to generate an enzyme-deficient C3, labelled with two molecules of the fluorescence dye. Alexa-labeled C3-bot is used for cell binding experiments to study and identify the receptor, through which C3-bot exerts its neurotrophic effects. The change in protein expression of primary cultured mouse hippocampal neurons, treated without or with C3-bot, is tested by DNA array technique. The difference in expression should help to identify those signal pathways that are used by cell receptors recruited by C3-bot. Thus, the identification of the signal cascade will allow to pinpoint the neuronal C3-receptor.

Knowledge about ligand (C3-bot derived peptide) receptor and the down stream signals may lead to the generation of diagnostic tools also applicable in vivo by which changes in the amount of these receptors can be estimated.

Equivalents: Those skilled in the art will recognize, or be able to ascertain using no more than routine experimentation, many equivalents to the specific embodiments of the invention described herein. Such equivalents are intended to be encompassed by the following claims.

### DESCRIPTION OF FIGURES

FIG. 1 C3-bot enhances axonal growth and branching when applied at nanomolar concentrations

Purified C3-bot was applied to hippocampal neurons at 20 nM for 5 days. Panel A gives the comparison between control neurons stained by an antibody against neurofilament protein (200 kDa) and a C3-bot treated neuron. Panel B shows the data of axon length and number of axon branches which have been combined from 82 neurons (control) or 73 neuron (C3-bot-treated). C3-bot increased the axonal length and number of branches. Significance according to students T-test (two-tailed type 3) denoted by a star revealed the following p-values p=1.1xE-20 for axonal length and p=7.5E-5 for branching).

FIG. 2 Photomicrographs of hippocampal neurons treated with 20 nM of various recombinant C3 proteins

The indicated rec. C3 proteins and mutant proteins were applied in a concentration of 20 nM for 5 days. From the various C3 proteins with ADP-ribosylating activity (rec. C3-bot; rec. C3-lim; rec. C3-stau2) tested, exclusively rec. C3-bot showed the neurotrophic property. This neurotrophic property was still present in the enzymatic-deficient (i. e. non-rho-ADP-ribosylating) C3-botE174A (SEQ ID NO: 2) and C3-botE174Q (SEQ ID NO: 1) proteins Thus, only rec. C3-bot and its enzymatic-deficient forms C3-botE174A and C3-botE174Q are capable of promoting axonal growth and branching.

FIG. 3 Combined quantitative analysis of the effects on axon length and branching in neurons after treatment with various recombinant C3 proteins

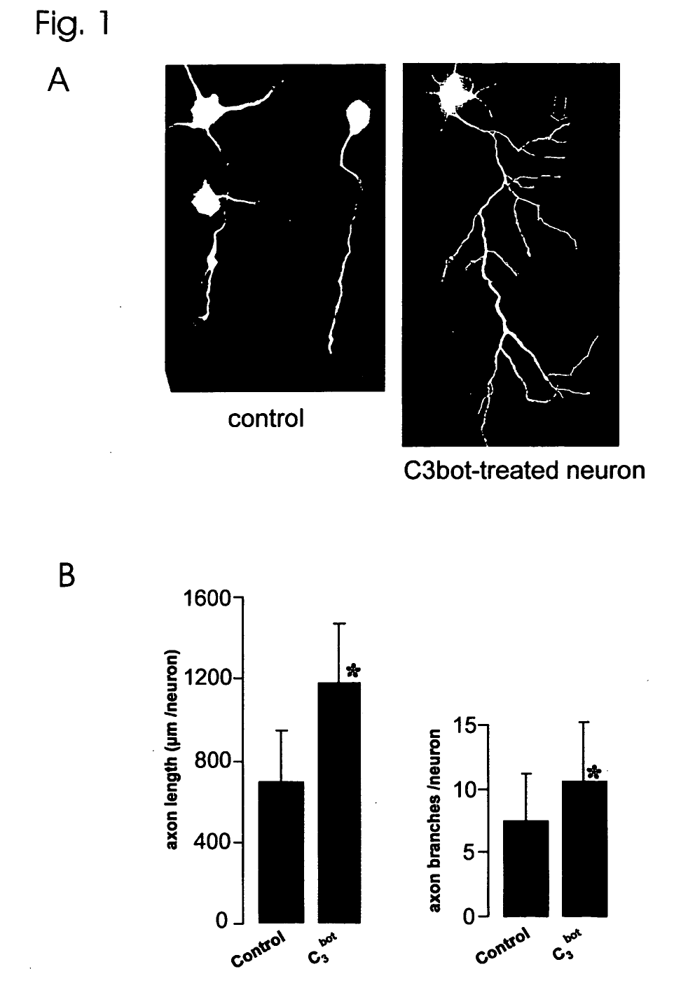

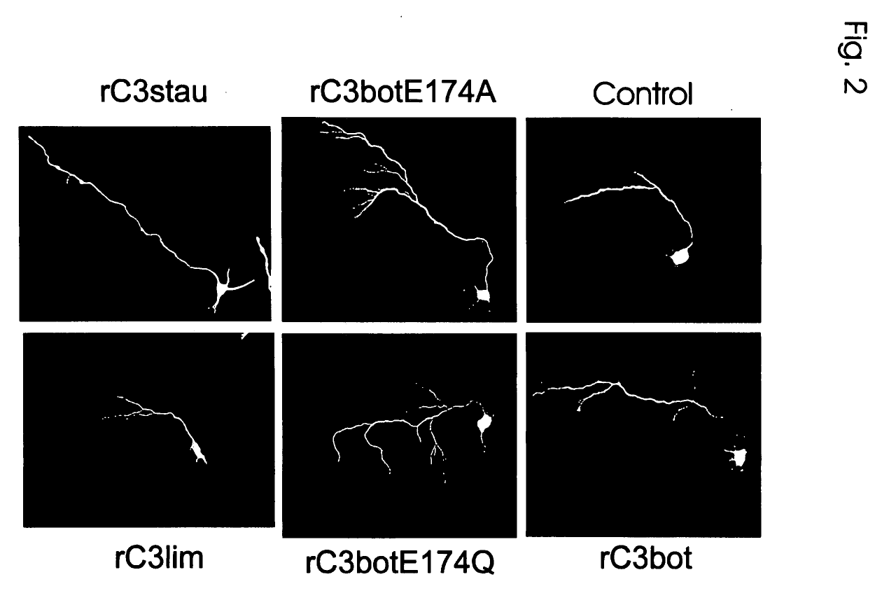

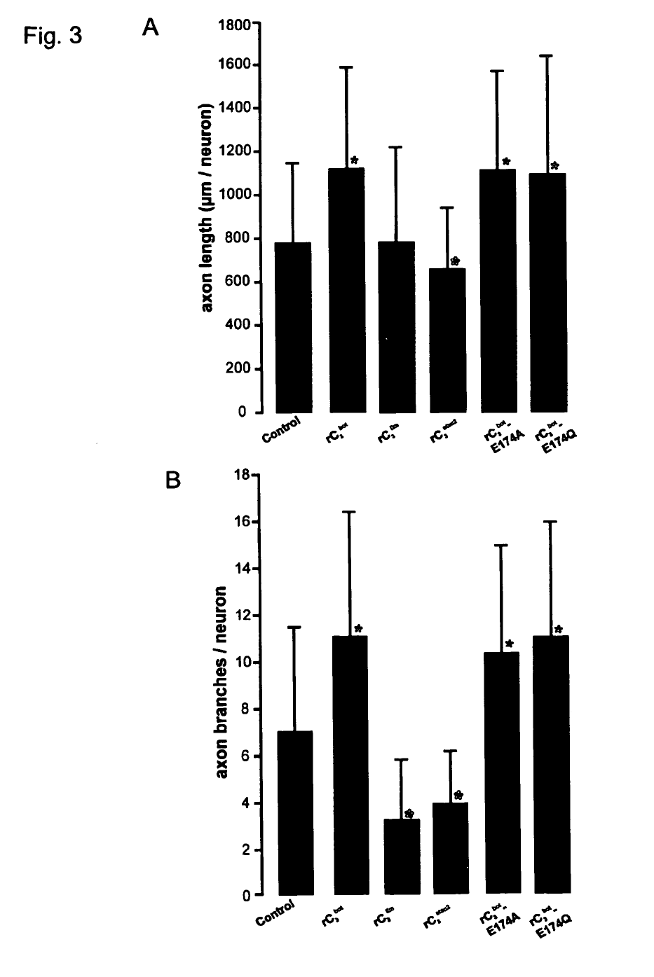

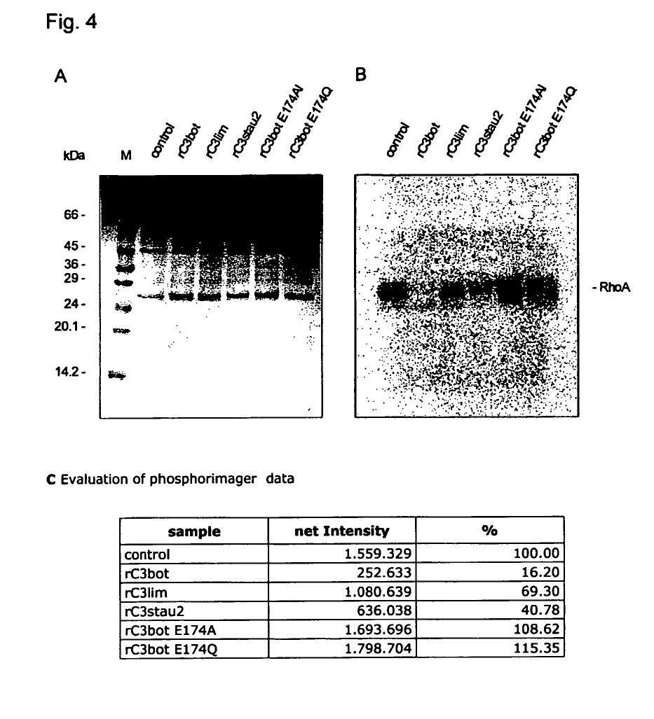

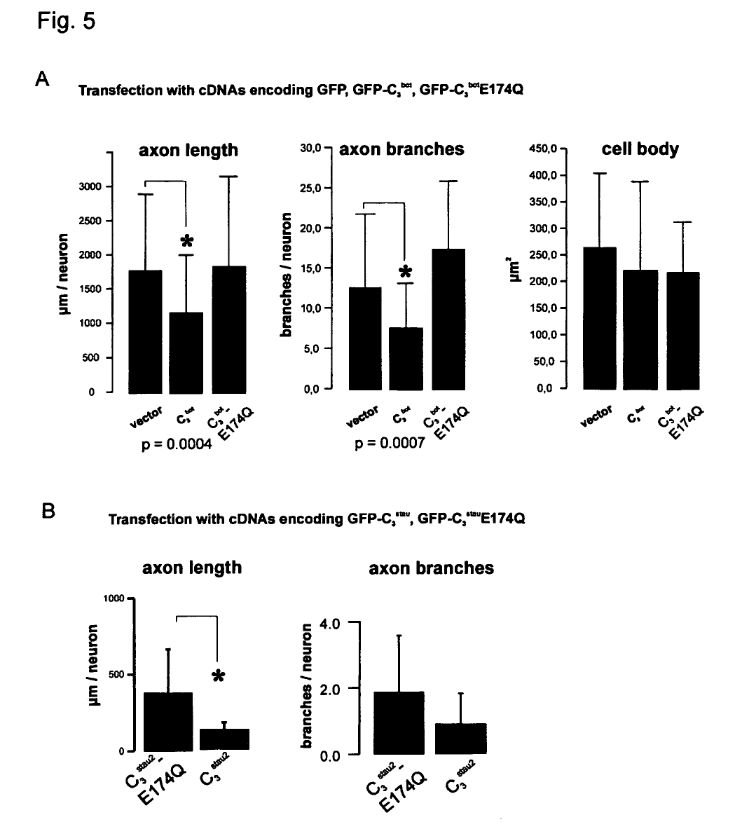

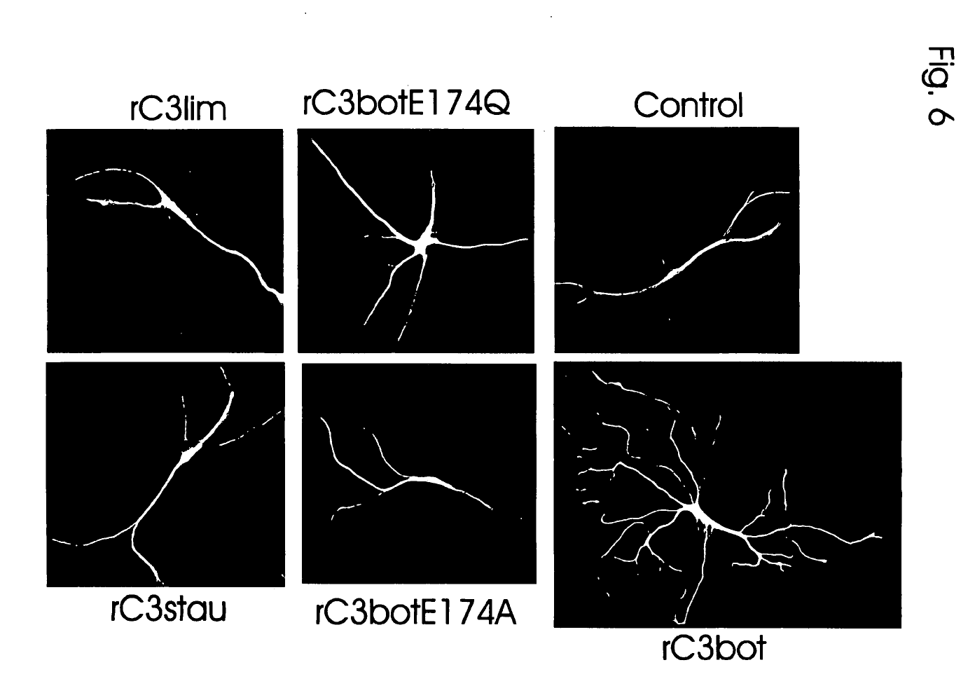

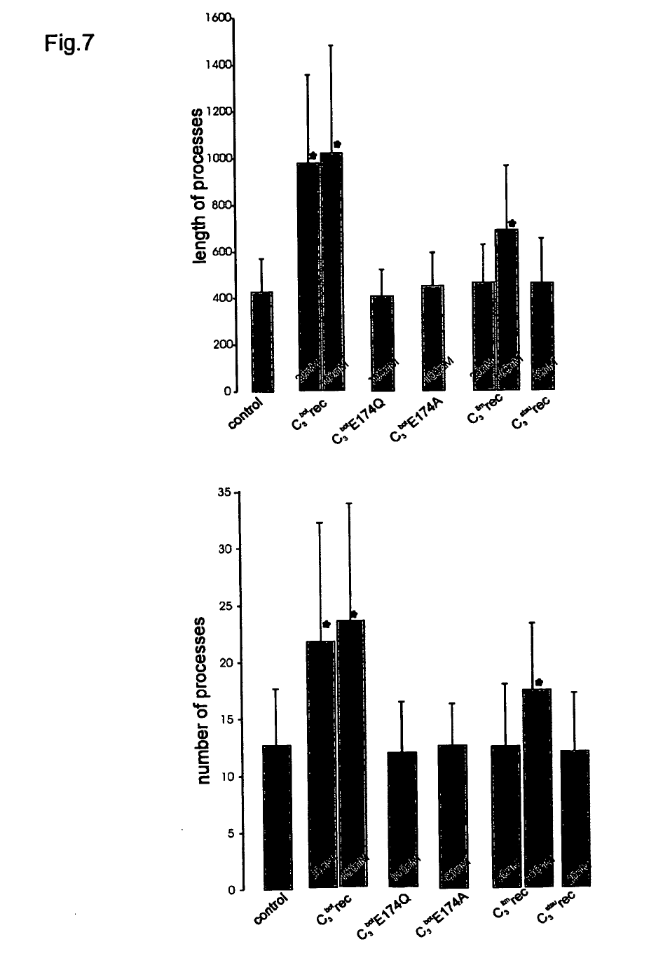

The graphs give the combined morphometric analyses obtained from a variety of preparations (see below) for the effects of 20 nM of each C3 protein on axon length (A) and axon branches (B). The following number of neurons were analysed for each condition: control: 256 neurons from 11 preparations; rec. C3-bot: 142 neurons from 4 preparations; rec. C3-lim: 59 neurons from 5 preparations; rec. C3-stau2: 29 neurons from 2 preparations; rec. C3-botE174A: 147 neurons from 4 preparations; rec. C3-botE174Q: 75 neurons from 3 preparations. Only the rec. C3-bot and the two enzymatic-deficient proteins increased axonal growth and branching. The respective p-values calculated against control are for axonal length: rec. C3-bot: 1.3E-12; C3-botE174A: 7.2E-13; C3-botE174Q: 7.2E-6: In contrast, the related proteins rec. C3-lim and rec. C3-stau3 had no effect or rather slightly decreased axonal growth (p-values rec. C3-lim: 0.9 and rec. C3-stau2: 0.04). A similar picture emerged when analysing axonal branching. Again only rec. C3-bot and the two enzymatic-deficient proteins significantly increased the number of branches with the following p-values calculated against controls: rec. C3-bot: 3E-13; C3-botE174A: 5.6E-11; C3-botE174Q: 8.2E-9. As with axonal length branching was significantly decreased by the rec. C3-lim (p=2.4E-14) and rec. C3-stau (p=5.8E-8).

FIG. 4 Differential ADP-ribosylation of hippocampal cultures pretreated with C3 proteins

Hippocampal primary cultures were incubated without any addition (control) or with rec. C3-bot, rec. C3-lim, rec. C3-stau2, C3-botE174A and C3-botE174Q (each 20 nM corresponding to 480 ng/ml 5 days. Thereafter, cultured cells were washed, homogenized and the post-nuclear supernatant was prepared by centrifugation. The supernatant (10 μg) was ADP-ribosylated by C3-bot in the presence of [P-32]NAD. The samples were separated on 15% SDS-PAGE and analysed by phosphorimager. The rational of this assay is that a decrease in incorporation of radioactivity in a protein band reflects the in vivo ADP-ribosylation in the intact cell. Panel A: SDS-PAGE of the samples, panel B: phosphorimager data, panel C: evaluation of phosphorimager data.

Enzymatically active C3-isoforms (C3-bot, C3-lim, C3-stau2) are capable of ADP-ribosylating intracellular Rho proteins, whereby C3-bot is the most efficacious one. The enzymatically inactive C3-bot mutants (C3-botE174A and C3-botE174Q) did not result in a decreased differential ADP-ribosylation indicating enzyme deficiency also under in vivo conditions.

FIG. 5 Intracellular effects of C3 proteins expressed in neurons after transfection with pEGFP-vector

C3 proteins are expressed as fusion proteins with the green fluorescent protein (GFP).

A: Morphometric analysis of neurons transfected with cDNAs encoding either GFP alone or GFP-C3-bot or GFP-C3-botE174Q each cloned in the EGFP vector. 20 neurons from each group obtained in two different preparations were analysed. A significant decrease against vector control was observed with the ADP-ribosylating GFP-C3-bot (p=0.004 for axon length and 0.0007 for branching) whereas transfection with the enzymatically inactive GFP-C3-botE174Q had no effect on the morphology of the neurons. B: Morphometric analysis of neurons transfected with cDNA encoding either enzymatic active GFP-C3-stau2 (12 neurons in 3 preparations) or its enzymatically inactive mutant GFP-C3-stau2E174Q (20 neurons in 3 preparations). The morphometric comparison is between the enzymatic active and inactive form, both cloned in the same vector. The enzymatic active C3-stau2 that inhibits Rho decreased axonal length (p=0.001) and axon branching (p=0.04) compared to the respective non-ADP-ribosylating C3-stau2E174Q.

FIG. 6 Photomicrographs of hippocampal astrocytes treated with 20 nM of various recombinant C3 proteins

Astrocytes in hippocampal primary cultures were specifically stained with an antibody against the glial fibrillary acidic protein (GFAP). The indicated rec. C3 proteins were applied with a concentration of 20 nM for 5 days. The comparison of the various C3 proteins with either ADP-ribosylating activity (rC3-bot; rC3-lim; and rC3-stau2) shows that at these concentrations only rec. C3-bot promoted morphological changes in these cells whereas the enzymatically inactive C3-botE174A and C3-botE174Q proteins showed no effect.

FIG. 7 Combined quantitative analysis of the effects of various recombinant C3 proteins on length and number of processes in astrocytes

The graphs give the combined morphometric analyses obtained with a variety of preparatins (see below) for the effects of 20 or 100 nM C3 proteins on length (A) and number (B) of astrocyte processes. The following number of astrocytes were analysed for each condition: control: 115 in 4 preparations; rec. C-bot (20 nM): 89 in 3 preparations; rec. C3-bot (100 nM): 55 in 2 preparations; C3botE174Q (100 nM): 43 in 2 preparations; C3botE174A (100 nM): 120 in 4 preparations; lim (20 nM): 89 in 3 preparations; rec. C3-lim (100 nM): 19 in 1 preparation; rec. C3-stau2 (20 nM): 36 in 1 preparations. Exclusively the enzymatic active rec. C3-bot and to a lesser extent rec. C3-lim promoted the stellation of astrocytes whereas the enzymatic-deficient C3-botE174A and C3botE174Q even at higher concentrations were inactive. The respective p-values calculated against control are the following:
rec. C3-bot (20 nM): 1.4E-23; rec. C3-bot (100 nM): 5.1E-13; rec. C3-lim (100 nM): 8.1E-6

1. An isolated nucleotide sequence selected from the group consisting of:
(a) a nucleotide sequence encoding a polypeptide having neurite outgrowth activity comprising a amino acid sequence of SEQ ID NO: 1-13;
(b) nucleotide sequences that are complementary to any of the nucleotide sequences of (a);
(c) a nucleotide sequence differing from the nucleotide sequence as claimed in (a) or (b) in codon sequences due to the degeneracy of the genetic code, whereby said nucleotide sequence encodes a polypeptide having a biological activity indicated in (a);
(d) a nucleotide sequence which specifically hybridize under stringent hybridization conditions to any of the nucleotide sequences of (a), (b) or (c);
(e) a nucleotide sequence of (a), (b), (c), or (d) having a deletion, addition, substitution mutation, whereby said nucleotide sequence encodes a polypeptide having a biological activity indicated in (a).

2. The isolated nucleotide sequence of claim 1 which is genomic DNA, cDNA or RNA.

3. A vector comprising the isolated nucleotide sequence of claim 1.

4. A host cell transfected with the vector of claim 3.

5. A polypeptide which promotes neurite outgrowth in mammals, selected from the group consisting of:
(a) a polypeptide having an amino acid sequences corresponding to SEQ ID NO: 1-13;
(b) (b) a polypeptide having an amino acid sequence which has at least 40 % homology with the amino sequences of SEQ ID NO: 1-13;
(c) (c) a variant of a polypeptide of (a) or (b) containing plurality of said amino acid sequences;
(d) a conservatively substituted variant of a polypeptide of (a), (b) or (c) comprising a substitution, deletion, and/or insertion of one or more amino acids; or
(e) a functionally equivalent homologue of (a), (b), (c) or (d).

6. The polypeptide of claim 5, having an amino acid sequence which has at least 95 homology with one of the amino acid sequences of SEQ ID NO. 1-13.

7. A recognition agent capable of recognizing: an isolated nucleotide sequence selected from the group consisting of:
(a) a nucleotide sequence encoding a polypeptide having neurite outgrowth activity comprising a amino acid sequence of SEQ ID NO: 1-13;
(b) nucleotide sequences that are complementary to any of the nucleotide sequences of (a):
(c) a nucleotide sequence differing from the nucleotide sequence as claimed in (a) or (b) in codon sequences due to the degeneracy of the genetic code, whereby said nucleotide sequence encodes a polypeptide having a biological activity indicated in (a);
(d) a nucleotide sequence which specifically hybridize under stringent hybridization conditions to any of the nucleotide sequences of (a), (b) or (c);
(e) a nucleotide sequence of (a), (b), (c), or (d) having a deletion, addition, substitution mutation, whereby said nucleotide sequence encodes a polypeptide having a biological activity indicated in (a);
a polypeptide which promotes neurite outgrowth in mammals, selected from the group consisting of:
(a) a polypeptide having an amino acid sequences corresponding to SEQ ID NO: 1-13;
(b) a polypeptide having an amino acid sequence which has at least 40% homology with the amino sequences of SEQ ID NO: 1-13;
(c) a variant of a polypeptide of (a) or (b) containing plurality of said amino acid sequences;
(d) a conservatively substituted variant of a polypeptide of (a), (b) or (c) comprising a substitution, deletion, and/or insertion of one or more amino acids; or
(e) a functionally equivalent homologue of (a), (b), (c) or (d).

8. The recognition agent according to claim 7, whereby the agent is an antibody or an antisense-construct.

9. A pharmaceutical composition comprising a therapeutically effective amount of a nucleotide sequence selected from the group consisting of:
(a) a nucleotide sequence encoding a polypeptide having neurite outgrowth activity comprising a amino acid sequence of SEQ ID NO: 1-13;
(b) nucleotide sequences that are complementary to any of the nucleotide sequences of (a);
(c) a nucleotide sequence differing from the nucleotide sequence as claimed in (a) or (b) in codon sequences due to the degeneracy of the genetic code, whereby said nucleotide sequence encodes a polypeptide having a biological activity indicated in (a);
(d) a nucleotide sequence which specifically hybridize under stringent hybridization conditions to any of the nucleotide sequences of (a), (b) or (c);
(e) a nucleotide sequence of (a), (b) (c), or (d) having a deletion, addition, substitution mutation, whereby said nucleotide sequence encodes a polypeptide having a biological activity indicated in (a);
a polypeptide which promotes neurite outgrowth in mammals, selected from the group consisting of:
(a) a polypeptide having an amino acid sequences corresponding to SEQ ID NO: 1-13;
(b) a polypeptide having an amino acid sequence which has at least 40% homology with the amino sequences of SEQ ID NO: 1-13;
(c) a variant of a polypeptide of (a) or (b) containing plurality of said amino acid sequences;
(d) a conservatively substituted variant of a polypeptide of (a), (b) or (c) comprising a substitution, deletion, and/or insertion of one or more amino acids; or
(e) a functionally equivalent homologue of (a), (b), (c) or (d); and/or
an recognition agent of claim 7;
and one or more pharmaceutically acceptable adjuvant, excipient, carrier, buffer, diluent and/or customary pharmaceutical auxiliary.

10. A neurite outgrowth-promoting apparatus comprising a bioabsorbable matrix and an effective amount of a pharmaceutical composition of claim 9.

11. A kit for screening a molecule that binds a nucleotide sequences, a polypeptide, and/or a recognition agent, comprising:
a nucleotide sequence selected from the group consisting of:
(a) a nucleotide sequence encoding a polypeptide having neurite outgrowth activity comprising a amino acid sequence of SEQ ID NO: 1-13;
(b) nucleotide sequences that are complementary to any of the nucleotide sequences of (a);
(c) a nucleotide sequence differing from the nucleotide sequence as claimed in (a) or (b) in codon sequences due to the degeneracy of the genetic code, whereby said nucleotide sequence encodes a polypeptide having a biological activity indicated in (a);
(d) a nucleotide sequence which specifically hybridize under stringent hybridization conditions to any of the nucleotide sequences of (a), (b) or (c);
(e) a nucleotide sequence of (a), (b), (c), or (d) having a deletion, addition, substitution mutation, whereby said nucleotide sequence encodes a polypeptide having a biological activity indicated in (a);
a polypeptide which promotes neurite outgrowth in mammals, selected from the group consisting of:
(a) a polypeptide having an amino acid sequences corresponding to SEQ ID NO: 1-13;
(b) a polypeptide having an amino acid sequence which has at least 40% homology with the amino sequences of SEQ ID NO: 1-13;
(c) a variant of a polypeptide of (a) or (b) containing plurality of said amino acid sequences;
(d) a conservatively substituted variant of a polypeptide of (a), (b) or (c) comprising a substitution, deletion, and/or insertion of one or more amino acids: or
(e) a functionally equivalent homologue of (a), (b), (c) or (d);
or a recognition agent of claim 7 or 8 and/or a pharmaceutical composition of claim 9.

12. Use of a nucleotide sequence selected from the group consisting of:
(a) a nucleotide sequence encoding a polypeptide having neurite outgrowth activity comprising a amino acid sequence of SEQ ID NO: 1-13;
(b) nucleotide sequences that are complementary to any of the nucleotide sequences of (a);
(c) a nucleotide sequence differing from the nucleotide sequence as claimed in (a) or (b) in codon sequences due to the degeneracy of the genetic code, whereby said nucleotide sequence encodes a polypeptide having a biological activity indicated in (a);
(d) a nucleotide sequence which specifically hybridize under stringent hybridization conditions to any of the nucleotide sequences of (a), (b) or (c);
(e) a nucleotide sequence of (a), (b), (c), or (d) having a deletion, addition, substitution mutation, whereby said nucleotide sequence encodes a polypeptide having a biological activity indicated in (a);
a polypeptide which promotes neurite outgrowth in mammals, selected from the group consisting of:
(a) a polypeptide having an amino acid sequences corresponding to SEQ ID NO: 1-13;
(b) a polypeptide having an amino acid sequence which has at least 40% homology with the amino sequences of SEQ ID NO: 1-13;
(c) a variant of a polypeptide of (a) or (b) containing plurality of said amino acid sequences;
(d) a conservatively substituted variant of a polypeptide of (a), (b) or (c) comprising a substitution, deletion, and/or insertion of one or more amino acids; or
(e) a functionally equivalent homologue of (a), (b), (c) or (d) according to claim 5 or 6,; or
a recognition agent of claim 7 or to promote neural growth.

13. Use of a nucleotide sequence selected from the group consisting of:
(a) a nucleotide sequence encoding a polypeptide having neurite outgrowth activity comprising a amino acid sequence of SEQ ID NO: 1-13;
(b) nucleotide sequences that are complementary to any of the nucleotide sequences of (a);
(c) a nucleotide sequence differing from the nucleotide sequence as claimed in (a) or (b) in codon sequences due to the degeneracy of the genetic code, whereby said nucleotide sequence encodes a polypeptide having a biological activity indicated in (a);
(d) a nucleotide sequence which specifically hybridize under stringent hybridization conditions to any of the nucleotide sequences of (a), (b) or (c);
(e) a nucleotide sequence of (a), (b), (c), or (d) having a deletion, addition, substitution mutation, whereby said nucleotide sequence encodes a polypeptide having a biological activity indicated in (a);
a polypeptide which promotes neurite outgrowth in mammals, selected from the group consisting of:
(a) a polypeptide having an amino acid sequences corresponding to SEQ ID NO: 1-13;
(b) a polypeptide having an amino acid sequence which has at least 40% homology with the amino sequences of SEQ ID NO: 1-13;
(c) a variant of a polypeptide of (a) or (b) containing plurality of said amino acid sequences;
(d) a conservatively substituted variant of a polypeptide of (a), (b) or (c) comprising a substitution, deletion, and/or insertion of one or more amino acids, or
(e) a functionally equivalent homologue of (a), (b), (c) or (d); or
a recognition agent of claim 7 9 for the manufacture of an agent for diagnosis, prophylactic and/or therapeutic treatment of a damage of the central and/or peripheral nervous system

14. Use according to claim 13 for modulating neurite outgrowth of central and/or peripheral nervous system neurons in vitro or in vivo comprising contacting the neurons with nucleotide sequence, the polypeptide, the recognition agent and/or the pharmaceutical composition such that neurite outgrowth is modulated.

15. Use according to claim 13 for inducing neurite outgrowth in the central and/or peripheral nervous system of a patient with damage to the central and/or peripheral nervous system comprising administering to the patient the pharmaceutical composition.

16. Use according to claim 15 in which the damage is due to infarction, traumatic injury, surgical lesion or a degenerative disorder of the central nervous system

17. Use according to claim 15 in which the damage has occurred to the spinal cord.

18. Use according to claim 16 in which the degenerative disorder is selected from the group consisting of Parkinson's disease, amyotrophic lateral sclerosis, Alzheimer's disease, diffuse cerebral cortical atrophy, Lewy-body dementia, Pick disease, mesolimbocortical dementia, thalamic degeneration, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, HallervordenSpatz disease, Meige syndrome, familial tremors, Gilles de la Tourette syndrome, acanthocytic chorea, Friedreich ataxia, Holmes familial cortical cerebellar atrophy, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, and/or ophthalmoplegia.

19. Use of a nucleotide sequence selected from the group consisting of:
(a) a nucleotide sequence encoding a polypeptide having neurite outgrowth activity comprising a amino acid sequence of SEQ ID NO: 1-13;
(b) nucleotide sequences that are complementary to any of the nucleotide sequences of (a);
(c) a nucleotide sequence differing from the nucleotide sequence as claimed in (a) or (b) in codon sequences due to the degeneracy of the genetic code, whereby said nucleotide sequence encodes a polypeptide having a biological activity indicated in (a);
(d) a nucleotide sequence which specifically hybridize under stringent hybridization conditions to any of the nucleotide sequences of (a), (b) or (c);
(e) a nucleotide sequence of (a), (b), (c), or (d) having a deletion, addition, substitution mutation, whereby said nucleotide sequence encodes a polypeptide having a biological activity indicated in (a);
a polypeptide which promotes neurite outgrowth in mammals, selected from the group consisting of:
(a) a polypeptide having an amino acid sequences corresponding to SEQ ID NO: 1-13;
(b) a polypeptide having an amino acid sequence which has at least 40% homology with the amino sequences of SEQ ID NO: 1-13;
(c) a variant of a polypeptide of (a) or (b) containing plurality of said amino acid sequences;
(d) a conservatively substituted variant of a polypeptide of (a), (b) or (c) comprising a substitution deletion, and/or insertion of one or more amino acids; or
(e) a functionally equivalent homologue of (a), (b), (c) or (d); or a recognition agent of claim 7 to promote neural growth to induce a expansion and/or differentiation of stem cells.

20. Use of a nucleotide sequence selected from the group consisting of:
(a) a nucleotide sequence encoding a polypeptide having neurite outgrowth activity comprising a amino acid sequence of SEQ ID NO: 1-13;
(b) nucleotide sequences that are complementary to any of the nucleotide sequences of (a);
(c) a nucleotide sequence differing from the nucleotide sequence as claimed in (a) or (b) in codon sequences due to the degeneracy of the genetic code, whereby said nucleotide sequence encodes a polypeptide having a biological activity indicated in (a);
(d) a nucleotide sequence which specifically hybridize under stringent hybridization conditions to any of the nucleotide sequences of (a), (b) or (c);
(e) a nucleotide sequence of (a), (b), (c), or (d) having a deletion, addition, substitution mutation, whereby said nucleotide sequence encodes a polypeptide having a biological activity indicated in (a);
a polypeptide which promotes neurite outgrowth in mammals, selected from the group consisting of:
(a) a polypeptide having an amino acid sequences corresponding to SEQ ID NO: 1-13;
(b) a polypeptide having an amino acid sequence which has at least 40% homology with the amino sequences of SEQ ID NO: 1-13;
(c) a variant of a polypeptide of (a) or (b) containing plurality of said amino acid sequences;
(d) a conservatively substituted variant of a polypeptide of (a), (b) or (c) comprising a substitution, deletion, and/or insertion of one or more amino acids; or
(e) a functionally equivalent homologue of (a), (b), (c) or (d); or
a recognition agent of claim 7 to promote neural growth such that neurite outgrowth is modulated.

21. The method according to claim 20 for inducing neurite outgrowth in the central and/or peripheral nervous system of a patient with damage to the central and/or peripheral nervous system comprising administering to the patient the nucleotide sequence, the polypeptide, the recognition agent and/or the pharmaceutical composition.

22. The method according to claim 21 in which the damage is due to infarction, traumatic injury, surgical lesion or a degenerative disorder of the central and/or peripheral nervous system.

23. The method according to claim 21 in which the damage has occurred to the spinal cord.

24. The method according to claim 22 in which the degenerative disorder is selected from the group consisting of Parkinson's disease, amyotrophic lateral sclerosis, Alzheimer's disease, diffuse cerebral cortical atrophy, Lewy-body dementia, Pick disease, mesolimbocortical dementia, thalamic degeneration, Huntington chorea, cortical-striatal-spinal degeneration, cortical-basal ganglionic degeneration, cerebrocerebellar degeneration, familial dementia with spastic paraparesis, polyglucosan body disease, Shy-Drager syndrome, olivopontocerebellar atrophy, progressive supranuclear palsy, dystonia musculorum deformans, Hallervorden-Spatz disease, Meige syndrome, familial tremors, Gilles de la Tourette syndrome, acanthocytic chorea, Friedreich ataxia, Holmes familial cortical cerebellar atrophy, Gerstmann-Straussler-Scheinker disease, progressive spinal muscular atrophy, progressive balbar palsy, primary lateral sclerosis, hereditary muscular atrophy, spastic paraplegia, peroneal muscular atrophy, hypertrophic interstitial polyneuropathy, heredopathia atactica polyneuritiformis, optic neuropathy, and/or ophthalmoplegia.

25. A method for producing a polypeptide comprising culturing the host cell of claim 4 under conditions suitable for expression of a polypeptide encoded by a nucleotide sequence selected from the group consisting of:
(a) a nucleotide sequence encoding a polypeptide having neurite outgrowth activity comprising a amino acid sequence of SEQ ID NO: 1-13;
(b) nucleotide sequences that are complementary to any of the nucleotide sequences of (a);
(c) a nucleotide sequence differing from the nucleotide sequence as claimed in (a) or (b) in codon sequences due to the degeneracy of the genetic code, whereby said nucleotide sequence encodes a polypeptide having a biological activity indicated in (a);
(d) a nucleotide sequence which specifically hybridize under stringent hybridization conditions to any of the nucleotide sequences of (a), (b) or (c);
(e) a nucleotide sequence of (a), (b), (c), or (d) having a deletion, addition, substitution mutation, whereby said nucleotide sequence encodes a polypeptide having a biological activity indicated in (a); and
recovering the polypeptide from the cell culture.

26. A method for identifying a receptor/molecule that binds a polypeptide of claim 5 comprising the steps of: (a) providing a said polypeptide; (b) contacting the polypeptide with the candidate molecule; and (c) detecting binding of the candidate molecule to the polypeptide.
